(19) United States
(12) Patent Application Publication
(10) Pub. No.: US 2026/0354873 A1
(43) Pub. Date: Nov. 12, 2026

(54) BIODEGRADABLE METAL DEVICE FOR USE IN BONE

(71) Applicant: MiRus LLC, Marietta, GA (US)

(72) Inventors: Noah Roth, Marietta, GA (US); Jay Yadav, Atlanta, GA (US)

(21) Appl. No.: 19/748,447

(22) Filed: Jul. 21, 2026

Related U.S. Application Data
(63) Continuation-in-part of application No. 18/540,271, filed on Dec. 14, 2023.
(60) Provisional application No. 63/536,948, filed on Sep. 7, 2023.

(51) Int. Cl. A61B 17/72; A61L 27/58

(52) U.S. Cl. CPC A61B 17/7258; A61L 27/58

(57) ABSTRACT

A medical device for treating a fracture site in a fractured bone or for the fusing together of two adjacently positioned bone. The medical device includes a body portion formed of a metal material that is formulated to be degradable or bioabsorable in a patient after the medical device has been implanted in the patient.

### REFERENCED APPLICATIONS

The present application is a continuation-in-part of U.S. patent application Ser. No. 18/540,271 filed Dec. 14, 2023, which in turn claims priority to United States Provisional Application Ser. No. 63/536,948 filed Sep. 7, 2023, which is incorporated herein by reference.

### FIELD OF DISCLOSURE

The present disclosure relates generally to the fixation, repair, stabilization and/or fusion of bones and bone fractures. In one non-limiting arrangement, the present disclosure is directed to an medical device that can be partially or fully inserted into a fractured bone to facilitate in the fixation, repair and stabilization of the fractured bone, more particularly to an medical device that is partially or fully formed of a biodegradable or bioabsorbable metal that can be partially or fully inserted into a fractured bone to facilitate in the fixation, repair and stabilization of the fractured bone, and still more particularly to an medical device that is partially or fully formed of a biodegradable or bioabsorbable metal that primarily contains molybdenum, magnesium or aluminum that can be partially or fully inserted into a fractured bone to facilitate in the fixation, repair and stabilization of the fractured bone. In one non-limiting arrangement, the present disclosure is directed to a medical device used in spinal fusion applications for the fusion of one or more bones in a spine, more particularly to a medical device that is partially or fully formed of a biodegradable or bioabsorbable metal used in spinal fusion applications for the fusion of one or more bones in a spine, still more particularly to a medical device that is partially or fully formed of a biodegradable or bioabsorbable metal that primarily contains molybdenum, magnesium or aluminum used in spinal fusion applications for the fusion of one or more bones in a spine, and still even more particularly to an medical device that is partially or fully formed of a biodegradable or bioabsorbable metal that primarily contains molybdenum, magnesium or aluminum used in spinal fusion applications for the fusion of one or more bones in a spine.

### BACKGROUND

The common methods of treating bone fractures ranges from the setting of the bone fracture to constraining the motion in the area of the bone fracture via a cast or wrap. Commonly, pins, screws, rods and cement are used to repair fracture bone. In some of these bone fracture treatments, the fractured bone is not properly stabilized, thereby resulting in potential misalignment of the fractured bone. These prior art bone fixation devices have several disadvantages. For instance, casting devices can result in a) prolonged immobilization of the fractures region, thus adding discomfort and inconvenience to the patient during the healing of the fracture bone, b) can result in muscle atrophy due to prolonged immobilization, thus requiring extended recover periods and potential physical therapy, c) can increase incidence of thrombosis, and d) can result in disuse syndrome. Some bone fixation devices currently use compression plates and screw devices to apply a compression force across the fracture site. Such fixation devices sometimes require a large surgical incision over the bone at the fracture site. The installing of the plates and screws usually a) requires the disturbance of the soft tissues overlying the fracture site, b) causes a disturbance of the fracture hematoma, c) is a highly invasive procedure, d) can result in s risk of vascular lesions, e) can require the use of extracortical hardware for later removal of the fixation device, and/or f) can result in the stripping of the periosteum of bone which can result in compromises to the blood supply of the fracture bone fragments. Another system for treating a bone fracture site includes intramedullary nailing. In such a procedure, one or more nails are inserted into the intramedullary canal of a fractured bone, usually through an incision located at either end of the bone. Intramedullary nailing can be advantages over external casting and other methods of fracture stabilization. However, there are several disadvantages associated with intramedullary nailing, namely a) the procedure is highly invasive, b) requires a large incision into the bone (e.g., about 15 mm), c) can result in cortical reaming, d) results in substantial or full bone marrow displacement, e) can result in a risk of pulmonary embolism, f) the reaming of the intramedullary canal and the placement of a nail without reaming can compromise the intramedullary blood supply to the bone fracture, g) the shape of nail can be non-confirming to the shape of the intramedullary canal thereby resulting in improper or difficult placement of the nail in the canal, and/or h) hardware migration can result during the repair of the bone fracture, thereby resulting in slower recovery times and improper fracture repair. Several prior art intramedullary nails are illustrated in U.S. Pat. Nos. 6,783,530; 6,551,321; 6,224,600; US 2009/0018542; US 2008/0255560; and US 2002/0032444, all of which are incorporated by reference herein.

Metal devices such as nails, screws, fusion implants and the like are commonly formed of metal material. These medical devices are used to hold spinal bones in place to enable bone growth and fusion of the spinal bones.

Metal containing medical devices that are used to repair bone fracture or facilitate in spinal fusion type remain in the patient for the life of the patient. The existence of metal materials in the bone can result in pain or discomfort during weather changes and can interfere with the full fission together of bone fragments or bones.

In view of the current prior art, there is a need in the art for a metal medical device that is effective in stabilizing a bone fracture site or facilitating in bone fusion, and which metal medical device eventually degrades and decomposes.

### SUMMARY OF THE DISCLOSURE

The present disclosure relates generally to the fixation, repair, stabilization and/or fusion together of bones and/or bone fractures. In one non-limiting arrangement, the present disclosure is directed to an medical device that can be partially or fully inserted into a fractured bone to facilitate in the fixation, repair and stabilization of the fractured bone, more particularly to an medical device that is partially or fully formed of a biodegradable or bioabsorbable metal that can be partially or fully inserted into a fractured bone to facilitate in the fixation, repair and stabilization of the fractured bone, and still more particularly to an medical device that is partially or fully formed of a biodegradable or bioabsorbable metal that primarily contains molybdenum, magnesium or aluminum that can be partially or fully inserted into a fractured bone to facilitate in the fixation, repair and stabilization of the fractured bone. In one non-limiting arrangement, the present disclosure is directed to a medical device used in spinal fusion applications for the fusion of one or more bones in a spine, more particularly to a medical device that is partially or fully formed of a biodegradable or bioabsorbable metal used in spinal fusion applications for the fusion of one or more bones in a spine, still more particularly to a medical device that is partially or fully formed of a biodegradable or bioabsorbable metal that primarily contains molybdenum, magnesium or aluminum used in spinal fusion applications for the fusion of one or more bones in a spine, and still even more particularly to an medical device that is partially or fully formed of a biodegradable or bioabsorbable metal that primarily contains molybdenum, magnesium or aluminum used in spinal fusion applications for the fusion of one or more bones in a spine.

In one non-limiting aspect of the present disclosure, the medical device is generally configured to be partially or fully inserted into a canal of a fractured bone, has a sufficient length to span the fracture site of the bone fracture. In one non-limiting embodiment, the medical device is an expandable medical device that is configured to be expandable in the canal of a fractured bone so as to conform to the internal surface of the bone canal. In another non-limiting embodiment, a guidewire is optionally provided for use with the expandable medical device. The guidewire can be used to facilitate in the insertion of the expandable medical device into the bone canal of the fractured bone. The guidewire can have a size and shape suitable for insertion into the bone canal, a length sufficient to span the fracture site of the fractured bone, and sufficient flexibility and support to guide the expandable medical device partially or fully through the bone canal of the fractured bone. In another non-limiting embodiment, a sheath is optionally provided for use with the expandable medical device. The sheath can be used to facilitate in the insertion of the expandable medical device into the bone canal of the fractured bone. The sheath can have a size and shape suitable for insertion into the bone canal, a length sufficient to span the fracture site of the fractured bone, and/or an elongate longitudinal cavity that is sized and shaped to receive the expandable medical device partially or fully through the cavity. In another non-limiting embodiment, a hardenable surgical fluid can optionally be used with the medical device. The hardenable surgical fluid, when used, provides additional support across the fracture site when used with the medical device. In one non-limiting configuration, the expandable medical device includes or is a stent. In another non-limiting configuration, the medical device is not expandable and optionally has the form of a nail, rod, or screw. In another non-limiting configuration, the medical device is may or may not be expandable and is configured to be inserted between two spinal bones to facilitate in the spinal fusion of the two bones.

In another and/or alternative non-limiting aspect of the disclosure, the medical device is partially or fully formed of a metal material that will degrade after implantation or be bioreabsorbable after implantation. In one non-limiting embodiment, the degradable and/or bioabsorbable metal used to partially or fully form the medical device forms 50 wt. % to 100 wt. % (and all values and ranges therebetween) of the medical device. In another non-limiting embodiment, the degradable and/or bioabsorbable metal used to partially or fully form the medical device has one or more properties of a) there is a weight loss of 0.01% to at least 5% (and all values and ranges therebetween) of the degradable and/or bioabsorbable metal after being implanted one year in a patient (e.g., human, mammal, etc.), b) there is a weight loss of 0.05% to at least 8% (and all values and ranges therebetween) of the degradable and/or bioabsorbable metal after being implanted two years in a patient (e.g., human, mammal, etc.), and/or c) there is a weight loss of 0.1% to at least 10% (and all values and ranges therebetween) of the degradable and/or bioabsorbable metal after being implanted three years in a patient (e.g., human, mammal, etc.). In one non-limiting objective, the weight percent of the degradable and/or bioabsorbable metal decreases from the original weight at the time of implantation by at least 30% after ten (10) years of being implanted in a patient.

In another and/or alternative non-limiting aspect of the disclosure, the degradable and/or bioabsorbable metal that is used to partially or fully form the medical device includes a) a molybdenum material that includes 80 wt. % to 100 wt. % (and all values and ranges therebetween) molybdenum, b) a magnesium material that includes 50 wt. % to 98 wt. % (and all values and ranges therebetween) magnesium, c) an aluminum material that includes 80 wt. % to 95 wt. % (and all values and ranges therebetween) aluminum, or d) a zinc material that includes 80 wt. % to 99.9 wt. % (and all values and ranges therebetween) zinc. In one non-limiting embodiment, the degradable and/or bioabsorbable metal is formed of 95-100 wt. % (and all values and ranges therebetween) metals, and 0-5 wt. % (and all values and ranges therebetween) of a combined weight of carbon, oxygen, phosphorous, sulfur, hydrogen, nitrogen, carbides, nitrides, and compounds that include oxygen, nitrogen, hydrogen, phosphorous, sulfur, and/or carbon. In another non-limiting embodiment, the degradable and/or bioabsorbable metal used to partially or fully form the medical device includes 80 wt. % to 100 wt. % (and all values and ranges therebetween) of a magnesium material that includes 50 wt. % to 98 wt. % (and all values and ranges therebetween) magnesium. In one non-limiting formulation, the magnesium material includes 0.05 wt. % to 49.5 wt. % (and all values and ranges therebetween) one or more metals selected from the group consisting of aluminum, bismuth, boron, calcium, cobalt, copper, gallium, manganese, nickel, rare earth metal, tin, yttrium, zinc, and zirconium. In another non-limiting formulation, 90-100 wt. % (and all values and ranges therebetween) of the magnesium material includes metals selected from magnesium, aluminum, bismuth, boron, calcium, cobalt, copper, gallium, manganese, nickel, rare earth metal, tin, yttrium, zinc, and zirconium. In another non-limiting formulation, the magnesium material includes a) 0.5-15 wt. % (and all values and ranges therebetween) aluminum, b) 1-15 wt. % aluminum, c) 3-11 wt. % aluminum, or d) 5-11 wt. % aluminum. In another non-limiting formulation, the magnesium material includes a) 0.05-45 wt. % (and all values and ranges therebetween) bismuth, b) 0.05-35 wt. % bismuth, c) 0.4-0.7 wt. % bismuth, or d) 0.1-30 wt. % bismuth. In another non-limiting formulation, the magnesium material includes a) 0.0002-0.25 wt. % (and all values and ranges therebetween) boron, or b) 0.0002-0.04 wt. % boron. In another non-limiting formulation, the magnesium material includes a) 0.05-35 wt. % (and all values and ranges therebetween) calcium, b) 0.1-15 wt. % calcium, or c) 0.5-8 wt. % calcium. In another non-limiting formulation, the magnesium material includes a) 0.01-5 wt. % cobalt, b) 0.5-15 wt. % cobalt, c) 15-35 wt. % cobalt, or d) 0.01-20 wt. % cobalt (and all values and ranges therebetween). In another non-limiting formulation, the magnesium material includes a) 0.01-5 wt. % copper, b) 0.5-15 wt. % copper, c) 15-35 wt. % copper, or d) 0.01-20 wt. % copper (and all values and ranges therebetween). In another non-limiting formulation, the magnesium material includes a) 1-10 wt. % (and all values and ranges therebetween) gallium, b) 2-8 wt. % gallium, or c) 3.01-5 wt. % gallium. In another non-limiting formulation, the magnesium material includes a) 0.015-2 wt. % (and all values and ranges therebetween) manganese, b) 0.05-0.25 wt. % manganese, c) 0.05-1 wt. % manganese, or d) 0.1-0.8 wt. % manganese. In another non-limiting formulation, the magnesium material includes a) 0.05-23.5 wt. % nickel, b) 0.01-5 wt. % nickel, c) 3-7 wt. % nickel, d) 7-10 wt. % nickel, e) 10-24.5 wt. % nickel, or f) 0.01-24.5 wt. % (and all values and ranges therebetween) nickel. In another non-limiting formulation, the magnesium material includes a) 0.5-30 wt. % (and all values and ranges therebetween) tin, or b) 1-10 wt. % tin. In another non-limiting formulation, the magnesium material includes a) 0.05-6 wt. % (and all values and ranges therebetween), b) 0.1-6 wt. % zinc, c) 0.05-4 wt. % zinc, d) 0.1-3 wt. % zinc, or e) 0.1-2 wt. % zinc. In another non-limiting formulation, the magnesium material includes a) 0.01-3 wt. % (and all values and ranges therebetween) zirconium, b) 0.05-3 wt. % zirconium, c) 0.01-1 wt. % zirconium, or d) 0.1-0.8 wt. % manganese. In another non-limiting formulation, the aluminum material includes 5 wt. % to 20 wt. % (and all values and ranges therebetween) of one or more metals selected from the group consisting of bismuth, calcium, gallium, magnesium, rare earth metal, and tin. In another non-limiting formulation, 90-100 wt. % (and all values and ranges therebetween) of the aluminum material includes metals selected from aluminum, bismuth, calcium, gallium, magnesium, and tin. In another non-limiting formulation, the zinc material includes 0.1-20 wt. % (and all values and ranges therebetween) of one or more metals selected from the group consisting of bismuth, copper, calcium, lithium, magnesium, rare earth metal, silver, strontium, and tin. In another non-limiting formulation, 90-100 wt. % (and all values and ranges therebetween) of the zinc material includes metals selected from zinc, bismuth, copper, calcium, lithium, magnesium, rare earth metal, silver, strontium, and tin.

In accordance with another and/or alternative aspect of the present disclosure, there is provided medical device that is an expandable medical device and wherein at least a portion of the medical device is at least partially formed of a metal alloy and is configured to be radially collapsible to a collapsed or crimped state for introduction into at least a portion of a bone (optionally via a sheath or guidewire) and radially expandable to an expanded state for implanting the expandable medical device at a bone fracture site or bone fusion site.

In accordance with another and/or alternative aspect of the present disclosure, there is provided a medical device that can be optionally coated with a polymer material (e.g., silicone, PTFE, ePTFE, polyurethane, polyolefins, hydrogels, biological materials (e.g., pericardium or biological polymers such as collagen, gelatin, or hyaluronic acid derivatives), etc.). The coating can be used to partially or fully encapsulate the structures on the medical device and/or to fill in openings on the medical device.

In accordance with another and/or alternative aspect of the present disclosure, the medical device is generally designed to include at least about 5 wt. % (e.g., 5-100 wt. % and all values and ranges therebetween) of the degradable and/or bioreabsorable metal alloy.

In accordance with another and/or alternative aspect of the present disclosure, the medical device can optionally be formed from into a shape that is at least 80% (e.g., 80-100% and all values and ranges therebetween) of the final net shape of the medical device.

In accordance with another and/or alternative non-limiting aspect of the present disclosure, the medical device can optionally be partially (e.g., 1% to 99.99% and all values and ranges therebetween) or fully be coated with and/or include one or more agents. The term “agent” includes, but is not limited to a substance, pharmaceutical, biologic, veterinary product, drug, and analogs or derivatives otherwise formulated and/or designed to prevent, inhibit and/or treat one or more clinical and/or biological events, and/or to promote healing. Non-limiting examples of clinical events that can be addressed by one or more agents include, but are not limited to, viral, fungus and/or bacterial infection; vascular diseases and/or disorders; lymphatic diseases and/or disorders; cancer; implant rejection; pain; nausea; swelling; organ failure; immunity diseases and/or disorders; cell growth inhibitors, blood diseases and/or disorders; heart diseases and/or disorders; neuralgia diseases and/or disorders; fatigue; genetic diseases and/or disorders; trauma; cramps; muscle spasms; tissue repair; nerve repair; neural regeneration and/or the like. The type and/or amount of agent included coated on the medical device can vary. In accordance with another and/or alternative aspect of the present disclosure, one or more portions of the medical device can optionally 1) include the same or different agents, 2) include the same or different amount of one or more agents, 3) include the same or different polymer coatings, 4) include the same or different coating thicknesses of one or more polymer coatings, 5) have one or more portions of the medical device controllably release and/or uncontrollably release one or more agents, and/or 6) have one or more portions of the medical device controllably release one or more agents and one or more portions of the medical device uncontrollably release one or more agents.

In another and/or alternative non-limiting aspect of the disclosure, the medical device can optionally include a marker material that facilitates in enabling the medical device to be properly positioned at a treatment site. The marker material is typically designed to be visible to electromagnetic waves (e.g., x-rays, microwaves, visible light, infrared waves, ultraviolet waves, etc.); sound waves (e.g., ultrasound waves, etc.); magnetic waves (e.g., MRI, etc.); and/or other types of electromagnetic waves (e.g., microwaves, visible light, infrared waves, ultraviolet waves, etc.).

In accordance with another and/or alternative aspect of the present disclosure, the medical device can optionally include one or more surface structures (e.g., pore, channel, pit, rib, slot, notch, bump, teeth, needle, well, hole, groove, etc.). These structures can be at least partially formed by MEMS (e.g., micro-machining, etc.) technology and/or other types of technology. In one non-limiting embodiment, the outer surface of at least a portion of the medical device includes a plurality of ribs, bumps, teeth, and/or grooves that are used to engage an inner surface of intramedullary canal of the fractured bone so as to facilitate in anchoring at least a portion of the medical device in the intramedullary canal when the medical device is expanded in the intramedullary canal.

In accordance with another and/or alternative aspect of the present disclosure, the medical device can optionally include one or more micro-structures (e.g., micro-needle, micro-pore, micro-cylinder, micro-cone, micro-pyramid, micro-tube, micro-parallelopiped, micro-prism, micro-hemisphere, teeth, rib, ridge, ratchet, hinge, zipper, zip-tie like structure, etc.) on the surface of the medical device. As defined herein, a “micro-structure” is a structure having at least one dimension (e.g., average width, average diameter, average height, average length, average depth, etc.) that is no more than about 2 mm, and typically no more than about 1 mm.

In another and/or alternative aspect of the disclosure, the medical device can optionally be an expandable device that can be expanded by use of some other device (e.g., balloon, etc.). The medical device can be fabricated from a material that has no or substantially no shape-memory characteristics.

In accordance with another and/or alternative aspect of the present disclosure, there is optionally provided a near net process for the medical device. In one non-limiting embodiment of the disclosure, there is provided a method of powder pressing materials and increasing the strength post-sintering by imparting additional cold work. In one non-limiting embodiment, the green part is pressed and then sintered. Thereafter, the sintered part is again pressed to increase its mechanical strength by imparting cold work into the pressed and sintered part.

In accordance with another and/or alternative aspect of the present disclosure, the metal alloy used to at least partially form the medical device is initially formed into a blank, a rod, a tube, etc., and then finished into final form by one or more finishing processes. The metal alloy blank, rod, tube, etc., can be formed by various techniques such as, but not limited to, 1) melting the metal alloy and/or metals that form the metal alloy (e.g., vacuum arc melting, etc.) and then extruding and/or casting the metal alloy into a blank, rod, tube, etc., 2) melting the metal alloy and/or metals that form the metal alloy, forming a metal strip, and then rolling and welding the strip into a blank, rod, tube, etc., 3) consolidating the metal powder of the metal alloy and/or metal powder of metals that form the metal alloy into a blank, rod, tube, etc., or 4) 3-D printing the metal powder of the metal alloy and/or metal powder of metals that form the metal alloy into a blank, rod, tube, etc. When the metal alloy is formed into a blank, the shape and size of the blank is non-limiting.

In accordance with another and/or alternative aspect of the present disclosure, when the metal powder is consolidated to form the metal alloy into a blank, rod, tube, etc., the metal powder is pressed together to form a solid solution of the metal alloy into a near net medical device, near net component of an medical device, blank, rod, tube, etc. Typically, the pressing process is by an isostatic process (i.e., uniform pressure applied from all sides on the metal powder); however other processes can be used. When the metal powders are pressed together isostatically, cold isostatic pressing (CIP) is typically used to consolidate the metal powders; however, this is not required. The pressing process can be performed in an inert atmosphere, an oxygen-reducing atmosphere (e.g., hydrogen, argon and hydrogen mixture, etc.), and/or under a vacuum; however, this is not required.

In accordance with another and/or alternative aspect of the present disclosure, when the metal powder is used to 3D print an medical device, component of an medical device, blank, rod, tube, etc., the average particle size of the metal powder is optionally 2-62 microns, and more particularly about 5-49.9 microns, the average density of the metal powders is greater than 5g/cm 3 , and the metal powder is generally spherical-shaped, and the Hall flow (s/ 50 g) is less than 30 seconds (e.g., 2-29.99 seconds and all values and ranges therebetween).

In accordance with another and/or alternative non-limiting aspect of the present disclosure, the medical device can be partially (e.g., 1% to 99.99% and all values and ranges therebetween) or fully be coated with an enhancement coating to improve one or more properties of the medical device (e.g., change exterior color of material having coated surface, increase surface hardness by use of the coated surface, increase surface toughness material having coated surface, reduced friction via use of the coated surface, improve scratch resistance of material that has the coated surface, improve impact wear of coated surface, improve resistance to corrosion and oxidation of coated material, form a non-stick coated surface, improve biocompatibility of material having the coated surface, reduce toxicity of material having the coated surface, reduce ion release from material having the coated surface, the enhancement coating forms a surface that is less of an irritant to cell about the coated surface after the medical device is implanted, promote the formation of nitric oxide, etc.). Non-limiting enhancement coatings that can be applied to a portion or all of the medical device includes chromium nitride (CrN), diamond-like carbon (DLC), titanium nitride (TiN), titanium nitride oxide (TiNOx), titanium oxynitride (TiNxOy), zirconium nitride (ZrN), zirconium oxide (ZrO2), zirconium-nitrogen-carbon (ZrNC), zirconium OxyCarbide (ZrOC), zirconium oxynitride (ZrNxOy) [e.g., cubic ZrN:O, cubic ZrO2:N, tetragonal ZrO2:N, and monoclinic ZrO2:N phase coatings], and combinations of such coatings. In one non-limiting embodiment, the one or more enhancement coatings are optionally applied to a portion or all of the medical device by a vacuum process using an energy source to vaporize material and deposit a thin layer of enhancement coating material. Such vacuum coating process, when used, can include a physical vapor deposition (PVD) process (e.g., sputter deposition, cathodic arc deposition or electron beam heating, etc.), chemical vapor deposition (CVD) process, atomic layer deposition (ALD) process, or a plasma-enhanced chemical vapor deposition (PE-CVD) process. In one non-limiting embodiment, the coating process is one or more of a PVD, CVD, ALD and PE-CVD, and wherein the coating process occurs at a temperature of 200-400 °C (and all values and ranges therebetween) for at least 10 minutes (e.g., 10-400 minutes and all values and ranges therebetween). In another non-limiting embodiment, the coating process is one or more of a PVD, CVD, ALD and PE-CVD, and wherein the coating process occurs at a temperature of 220-300 °C for 60-120 minutes. In another non-limiting embodiment, when the materials of the one or more enhancement coatings are to be applied to the outer surface of the medical device that is partially or fully formed of a metal alloy, the materials of the one or more enhancement coatings can optionally be combine with one or more metals in the metal alloy, and/or combined with nitrogen, oxygen, carbon, or other elements that are in the metal alloy and/or present in the atmosphere about the metal alloy to a form an enhancement coating on the outer surface of the metal alloy. In another non-limiting embodiment, when the materials of the one or more enhancement coatings are to be applied to the outer surface of the medical device that is partially or fully formed of a metal alloy, the materials of the one or more enhancement coatings can optionally be used to form various coating colors on the outer surface of the metal alloy (e.g., gold, copper, brass, black, rose gold, chrome, blue, silver, yellow, green, etc.). In another non-limiting embodiment, the thickness of the enhancement coating is greater than 1 nanometer (e.g., 2 nanometers to 100 microns and all values and ranges therebetween), and typically 0.1-25 microns, and more typically 0.2-10 microns. In another non-limiting embodiment, the hardness of the enhancement coating can be at least 5 GPa (ASTM C1327-15 or ASTM C1624-05), typically 5-50 GPa (and all values and ranges therebetween), more typically 10-25 GPa, and still more typically 14-24 GPa. In another non-limiting embodiment, the coefficient of friction (COF) of the enhancement coating can be 0.04-0.2 (and all values and ranges therebetween), and typically 0.6-0.15. In another non-limiting embodiment, the wear rate of the enhancement coating can be 0.5×10-7 mm3/N-m to 3×10-7mm3/N-m (an all values and ranges therebetween), and typically 1.2×10-7 mm 3/N-m to 2×10-7mm3/N-m. In another non-limiting embodiment, silicon-based precursors (e.g., trimethysilane, tetramethylsilane, hexachlorodisilane, silane, dichlorosilane, trichlorosilane, silicon tetrachloride, tris(dimethylamino) silane, bis(tert-butylamino)silane, trisilylamine, allyltrimethoxysilane, (3-aminopropyl)triethoxysilane, butyltrichlorosilane, n-sec-butyl(trimethylsilyl)amine, chloropentamethyldisilane, 1,2-dichlorotetramethyldisilane, [3-(diethylamino)propyl]trimethoxysilane, 1,3-diethyl-1,1,3,3-tetramethyldisilazane, dimethoxydimethylsilane, dodecamethylcyclohexasilane, hexamethyldisilane, isobutyl(trimethoxy)silane, methyltrichlorosilane, 2,4,6,8,10-pentamethylcyclopentasiloxane, pentamethyldisilane, n-propyltriethoxysilane, silicon tetrabromide, silicon tetrabromide, etc.) can optionally be used to facilitate in the application of the enhancement coating to one or more portions or all of the medical device.

In accordance with another and/or alternative non-limiting aspect of the present disclosure, the medical device can be partially or fully coated with an enhancement coating composition that includes a chromium nitride (CrN) coating. A portion or all of the medical device can be partially or fully coated with the chromium nitride (CrN) coating. The enhancement coating can be used to improve hardness, improve toughness, reduced friction, resistant impact wear, improve resistance to corrosion and oxidation, and/or form a reduced stick surface when in contact with many different materials. In accordance with one non-limiting embodiment, the chromium nitride (CrN) coating generally includes 40-85 wt. % Cr (and all values and ranges therebetween), 15-60 wt. % N (and all values and ranges therebetween), 0-10 wt. % Re (and all values and ranges therebetween), 0-10 wt. % Si (and all values and ranges therebetween), 0-2 wt. % O (and all values and ranges therebetween), and 0-2 wt. % C (and all values and ranges therebetween). In one non-limiting coating process, all or a portion of the medical device are initially coated with Cr metal. The Cr metal coating can be applied by PVD, CVD, ALD and PE-CVD in an inert environment. The coating thickness of Cr metal is 0.5-15 microns. Thereafter, the Cr metal coating is exposed to nitrogen gas and/or a nitrogen containing gas compound to cause the nitrogen to react with the Cr metal coating to form a layer of CrN on the outer surface of the Cr metal coating and/or the outer surface of the medical device. Particles of Cr metal can optionally be mixed with nitrogen gas and/or a nitrogen containing gas compound to facilitate in the formation of the CrN coating. When Cr metal particles are used, the initial Cr coating layer on the medical device can optionally be eliminated. In another non-limiting embodiment, the enhancement coating composition generally includes 65-80 wt. % Cr, 15-30 wt. % N, 0-8 wt. % Re, 0-1 wt. % Si, 0-1 wt. % O, and 0-1 wt. % C.

In accordance with another and/or alternative non-limiting aspect of the present disclosure, the medical device can be partially or fully coated with an enhancement coating composition that includes a diamond-Like Carbon (DLC) coating. A portion or all of the medical device can be partially or fully coated with the diamond-Like Carbon (DLC) coating. The enhancement coating can be used to improve hardness, improve toughness, reduced friction, resistant impact wear, improve resistance to corrosion and oxidation, improve biocompatibility, and/or form a reduced stick surface when in contact with many different materials. In one non-limiting embodiment, the diamond-Like Carbon (DLC) coating generally includes 60-99.99 wt. % C (and all values and ranges therebetween), 0-2 wt. % N (and all values and ranges therebetween), 0-10 wt. % Re (and all values and ranges therebetween), 0 -20 wt. % Si (and all values and ranges therebetween), and 0-2 wt. % O (and all values and ranges therebetween). The carbon coating can be applied by PVD, CVD, ALD and PE-CVD in an inert environment. The carbon layer can be applied by use of methane and/or acetylene gas; however, other or additional carbon sources can be used. The coating thickness of the carbon is 0.5-15 microns. In another non-limiting embodiment, all or a portion of the medical device are coated with the enhancement coating composition that generally includes 90-99.99 wt. % C, 0-1 wt. % N, 0-8 wt. % Re, 0-1 wt. % Si, and 0-1 wt. % O.

In accordance with another and/or alternative non-limiting aspect of the present disclosure, the medical device can be partially or fully coated with an enhancement coating composition that includes a titanium nitride (TiN) coating. A portion or all of the outer surface of the medical device can include the titanium nitride (TiN) coating. The enhancement coating can be used to improve hardness, improve toughness, improve resistance to corrosion and oxidation, reduced friction, and/or form a reduced stick surface when in contact with many different materials. In one non-limiting embodiment, all or a portion of the outer surface of the medical device are optionally initially coated with Ti metal. The Ti metal coating, when applied, can be applied by PVD, CVD, ALD and PE-CVD in an inert environment. The coating thickness of Ti metal is 0.05-15 microns (and all values and ranges therebetween). As can be appreciated, the initial Ti coating is optional. Thereafter, the Ti metal coating, when applied, is exposed to nitrogen gas and/or a nitrogen containing gas compound and optionally titanium particles to cause the nitrogen to react with the Ti metal coating and/or titanium metal particles to form a layer of TiN on the outer surface of the Ti metal coating and/or the outer surface of the medical device. If a titanium layer is not preapplied, the TiN coating can be formed by exposing the medical device to titanium particles and nitrogen gas and/or a nitrogen containing gas compound. The coating thickness of the TiN coating is generally 0.1-15 microns (and all values and ranges therebetween), and typically 0.2-2 microns.

In accordance with another and/or alternative non-limiting aspect of the present disclosure, the medical device can be partially or fully coated with an enhancement coating composition that includes a titanium nitride oxide (TiNOx) or titanium oxynitride (TiNxOy), coating. A portion or all of the outer surface of the medical device can include the titanium nitride oxide (TiNOx) or titanium oxynitride (TiNxOy) coating. The enhancement coating can be used to improve hardness, improve toughness, improve resistance to corrosion and oxidation, reduced friction, and/or form a reduced stick surface when in contact with many different materials. In one non-limiting embodiment, all or a portion of the outer surface of the medical device are optionally initially coated with Ti metal. The Ti metal coating, when applied, can be applied by PVD, CVD, ALD and PE-CVD in an inert environment. The coating thickness of Ti metal is 0.05-15 microns (and all values and ranges therebetween). As can be appreciated, the initial Ti coating is optional. Thereafter, the Ti metal coating is exposed to titanium particles and a nitrogen and oxygen mixture that can include nitrogen gas, oxygen gas, a nitrogen containing gas compound and/or an oxygen containing gas compound to cause the nitrogen and oxygen to react with the Ti metal coating, if such coating is used, and/or with the Ti metal particles to form a layer of titanium nitride oxide (TiNOx) or titanium oxynitride (TiNxOy) on the outer surface of the Ti metal coating and/or the outer surface of the medical device. The ratio of the N to the O can be varied to control the about of O in the titanium nitride oxide (TiNOx) or titanium oxynitride (TiNxOy) coating. If a titanium layer is not preapplied, the titanium nitride oxide (TiNOx) or titanium oxynitride (TiNxOy) coating can be formed by exposing the medical device to titanium particles and a nitrogen and oxygen source such as nitrogen gas, oxygen gas, a nitrogen containing gas compound and/or an oxygen containing gas compound. The ratio of N to O when forming the titanium nitride oxide (TiNOx) or titanium oxynitride (TiNxOy) coating is generally 1:10 to 10:1 (and all values and ranges therebetween). The coating thickness of the titanium nitride oxide (TiNOx), titanium oxynitride (TiNxOy) coating is generally 0.1-15 microns (and all values and ranges therebetween), and typically 0.2-2 microns. In another non-limiting embodiment, a titanium nitride oxide (TiNOx) or titanium oxynitride (TiNxOy) coating is applied to a portion or all of the outer surface of the medical device, and the titanium nitride oxide (TiNOx) or titanium oxynitride (TiNxOy) coating is formed by a) exposing the outer surface of a portion of all of the medical device to Ti particles (PVD, CVD, ALD and PE-CVD process) and/or a Ti containing solution to form a Ti layer on a portion of all of the medical device, and wherein the thickness of the Ti coating is 0.05-5 microns, and b) exposing the Ti coating to a nitrogen and oxygen source such as nitrogen gas, oxygen gas, a nitrogen containing gas compound and/or an oxygen containing gas compound to form a titanium nitride oxide (TiNOx) or titanium oxynitride (TiNxOy) coating, and wherein ratio of N to O when forming the titanium nitride oxide (TiNOx) or titanium oxynitride (TiNxOy) coating is generally 1:10 to 10:1, and wherein the coating thickness of the TiNOx coating is 0.2-5 microns. In another non-limiting embodiment, a titanium nitride oxide (TiNOx) or titanium oxynitride (TiNxOy) coating is applied to a portion or all of the outer surface of the medical device, and the titanium nitride oxide (TiNOx) or titanium oxynitride (TiNxOy) coating is formed by exposing a portion or all of the outer surface of the medical device to Ti particles and a nitrogen and oxygen source such as nitrogen gas, oxygen gas, a nitrogen containing gas compound and/or an oxygen containing gas compound to form a titanium nitride oxide (TiNOx) or titanium oxynitride (TiNxOy) coating, and wherein ratio of N to O when forming the titanium nitride oxide (TiNOx) or titanium oxynitride (TiNxOy) coating is generally 1:10 to 10:1, and wherein the coating thickness of the titanium nitride oxide (TiNOx) or titanium oxynitride (TiNxOy) coating is 0.2-5 microns. In another non-limiting embodiment, the enhancement coating composition generally includes 20-85 wt. % Ti (and all values and ranges therebetween), 0.5-35 wt. % N (and all values and ranges therebetween), 0-10 wt. % Re (and all values and ranges therebetween), and 0.5-35 wt. % O (and all values and ranges therebetween). In another non-limiting embodiment, a coating of titanium nitride oxide (TiNOx) or titanium oxynitride (TiNxOy) was formed on the medical device by reactive physical vapor deposition in a vacuum chamber. Depending on the oxygen-nitrogen ratio during vapor deposition, a coating deposit of titanium nitride oxide (TiNOx) or titanium oxynitride (TiNxOy) with defined composition and resistivity can be coated on the outer surface of the medical device.

In accordance with another and/or alternative non-limiting aspect of the present disclosure, the medical device can be partially or fully coated with an enhancement coating composition that includes a zirconium nitride (ZrN) coating. The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the medical device is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to nitrogen gas and/or a nitrogen containing gas compound to cause the nitrogen to react with the Zn metal coating to form a layer of ZrN on the outer surface of the Zr metal coating and/or the outer surface of the medical device. Particles of Zr metal can optionally be mixed with nitrogen gas and/or a nitrogen containing gas compound to facilitate in the formation of the ZrN coating. When Zr metal particles are used, the initial Zr coating layer on the medical device can optionally be eliminated. The ZrN coating has been found to produce a gold colored enhancement coating color. In another non-limiting embodiment, the enhancement coating composition generally includes 35-90 wt. % Zr (and all values and ranges therebetween), 5-25 wt. % N (and all values and ranges therebetween), 0-10 wt. % Re (and all values and ranges therebetween), 0-20 wt. % Si (and all values and ranges therebetween), 0-2 wt. % O (and all values and ranges therebetween), and 0-2 wt. % C (and all values and ranges therebetween). In another non-limiting embodiment, the enhancement coating composition generally includes 80-90 wt. % Zr, 10-20 wt. % N, 0-8 wt. % Re, 0-1 wt. % Si, 0-1 wt. % O, and 0-1wt. % C.

In accordance with another and/or alternative non-limiting aspect of the present disclosure, the medical device can be partially or fully coated with an enhancement coating composition that includes a zirconium oxide (ZrO2) coating. The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the medical device is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to oxygen gas and/or oxygen containing gas compound to cause the oxygen to react with the Zn metal coating to form a layer of zirconium oxide (ZrO2) on the outer surface of the Zr metal coating and/or the outer surface of the medical device. Particles of Zr metal can optionally be mixed with the oxygen gas and/or an oxygen containing gas compound to facilitate in the formation of the ZrO2 coating. When Zr metal particles are used, the initial Zr coating layer on the medical device can optionally be eliminated. The zirconium oxide (ZrO2) coating has been found to produce a blue colored enhancement coating color. In another non-limiting embodiment, the enhancement coating composition generally includes 35-90 wt. % Zr (and all values and ranges therebetween), 10-35 wt. % O (and all values and ranges therebetween), 0-2 wt. % N (and all values and ranges therebetween), 0-10 wt. % Re (and all values and ranges therebetween), 0-20 wt. % Si (and all values and ranges therebetween), and 0-2 wt. % C (and all values and ranges therebetween). In another non-limiting embodiment, the enhancement coating composition generally includes 70-80 wt. % Zr, 20-30 wt. %, 0-1 wt. % N, 0-8 wt. % Re, 0-1wt. % Si, and 0-1 wt. % C.

In accordance with another and/or alternative non-limiting aspect of the present disclosure, the medical device can be partially or fully coated with an enhancement coating composition that includes both a zirconium oxide (ZrO2) coating and a zirconium nitride coating (ZrN). The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the metal alloy is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to a) both oxygen gas and/or oxygen containing gas compound and also to nitrogen gas and/or nitrogen containing gas compound, b) nitrogen gas and/or nitrogen containing gas compound and then to oxygen gas and/or oxygen containing gas compound, or c) oxygen gas and/or oxygen gas containing compound and then to nitrogen gas and/or nitrogen gas containing compound. The coating composition of the zirconium oxide (ZrO2) coating and the zirconium nitride coating (ZrN) are similar or the same as discussed above. As discussed above, Particles of Zr metal can optionally be mixed with the oxygen gas and/or an oxygen containing gas compound to facilitate in the formation of the ZrO2 coating and the nitrogen gas and/or nitrogen gas containing compound to facilitate in the formation of the ZrN coating. When Zr metal particles are used, the initial Zr coating layer on the medical device can optionally be eliminated.

In accordance with another and/or alternative non-limiting aspect of the present disclosure, the medical device can be partially or fully coated with an enhancement coating composition that includes a zirconium oxycarbide (ZrOC) coating. The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the metal alloy is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to a) both to oxygen gas and/or an oxygen containing gas compound and to carbon and/or a carbon containing gas compound (e.g., methane and/or acetylene gas), b) carbon and/or a carbon containing gas compound and then to oxygen gas and/or an oxygen containing gas compound, or c) oxygen gas and/or oxygen containing gas compound and then to carbon and/or carbon containing gas compound. Particles of Zr metal can optionally be mixed with the oxygen gas and/or an oxygen containing gas compound and the carbon and/or carbon containing gas compound to facilitate in the formation of the zirconium oxycarbide (ZrOC) coating. When Zr metal particles are used, the initial Zr coating layer on the medical device can optionally be eliminated. In another non-limiting embodiment, the enhancement coating composition generally includes 40-95 wt. % Zr (and all values and ranges therebetween), 5-25 wt. % O (and all values and ranges therebetween), and 10-40 wt. % C (and all values and ranges therebetween), 0-2 wt. % N (and all values and ranges therebetween), 0-10 wt. % Re (and all values and ranges therebetween), and 0 -20 wt. % Si (and all values and ranges therebetween). In another non-limiting embodiment, the enhancement coating composition generally includes 40-65 wt. % Zr, 5 -25 wt. % O, and 25-40 wt. % C, 0-1 wt. % N, 0-8 wt. % Re, and 0-1 wt. % Si.

In accordance with another and/or alternative non-limiting aspect of the present disclosure, one or more components of the medical device can be partially or fully coated with an enhancement coating composition that includes a zirconium oxynitride (ZrNxOy) [e.g., cubic ZrN:O, cubic ZrO2:N, tetragonal ZrO2:N, and monoclinic ZrO2:N phase coatings]. A portion or all of the outer surface of the one or more components of the medical device can include the zirconium oxynitride (ZrNxOy). The enhancement coating can be used to improve hardness, improve toughness, improve resistance to corrosion and oxidation, reduced friction, form a reduced stick surface when in contact with many different materials, and/or promote nitric oxide formation on the surface of the coating. In one non-limiting embodiment, all or a portion of the outer surface of the one or more components of the medical device are optionally initially coated with Zr metal. The Zr metal coating, when applied, can be applied by PVD, CVD, ALD and PE-CVD in an inert environment. The coating thickness of Zr metal is 0.05-15 microns (and all values and ranges therebetween). As can be appreciated, the initial Zr coating is optional. Thereafter, the Zr metal coating is exposed to zirconium particles and a nitrogen and oxygen mixture that can include nitrogen gas, oxygen gas, a nitrogen containing gas compound and/or an oxygen containing gas compound to cause nitrogen and oxygen to react with the Zr metal coating, if such coating is used, and/or with the Zr metal particles to form a layer of (ZrNxOy) on the outer surface of the Zr metal coating and/or the outer surface of the one or more components of the medical device. The ratio of the N to the O can be varied to control the about of O and N in the (ZrNxOy) coating. If a zirconium layer is not preapplied, the (ZrNxOy) coating can be formed by exposing the outer surface of one or more components of the medical device to zirconium particles and a nitrogen and oxygen source such as nitrogen gas, oxygen gas, a nitrogen containing gas compound and/or an oxygen containing gas compound. The ratio of N to O when forming the (ZrNxOy) coating is generally 1:10 to 10:1 (and all values and ranges therebetween). The coating thickness of the (ZrNxOy) coating is generally 0.1-15 microns (and all values and ranges therebetween), and typically 0.2-2 microns. In another non-limiting embodiment, a (ZrNxOy) coating is applied to a portion or all of the outer surface of the one or more components of the medical device, and the (ZrNxOy) coating is formed by a) exposing the outer surface of a portion of all of the one or more components of the medical device to Zr particles (PVD, CVD, ALD and PE-CVD process) and/or a Zr containing solution to form a Zr layer on a portion of all of the one or more components of the medical device, and wherein the thickness of the Zr coating is 0.05-5 microns, and b) exposing the Zr coating to a nitrogen and oxygen source such as nitrogen gas, oxygen gas, a nitrogen containing gas compound and/or an oxygen containing gas compound to form a ZrNxOy coating, and wherein ratio of N to O when forming the (ZrNxOy) coating is generally 1:10 to 10:1, and wherein the coating thickness of the (ZrNxOy) coating is 0.2-5 microns. In another non-limiting embodiment, a (ZrNxOy) coating is applied to a portion or all of the outer surface of the one or more components of the medical device, and the (ZrNxOy) coating is formed by exposing a portion or all of the outer surface of the one or more components of the medical device to Zr particles and a nitrogen and oxygen source such as nitrogen gas, oxygen gas, a nitrogen containing gas compound and/or an oxygen containing gas compound to form a (ZrNxOy) coating, and wherein ratio of N to O when forming the (ZrNxOy) coating is generally 1:10 to 10:1, and wherein the coating thickness of the (ZrNxOy) coating is 0.2-5 microns. In another non-limiting embodiment, the enhancement coating composition generally includes 20-85 wt. % Zr (and all values and ranges therebetween), 0.5-35 wt. % N (and all values and ranges therebetween), and 0.5-35 wt. % O (and all values and ranges therebetween). In another non-limiting embodiment, a coating of (ZrNxOy) was formed on one or more components of the medical device by reactive physical vapor deposition in a vacuum chamber. Depending on the oxygen-nitrogen ratio during vapor deposition, a coating deposit of (ZrNxOy) with defined composition and resistivity can be coated on the outer surface of the one or more components of the medical device.

In accordance with another and/or alternative non-limiting aspect of the present disclosure, the medical device can be partially or fully coated with an enhancement coating composition that includes a zirconium-nitrogen-carbon (ZrNC) coating. The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the medical device is initially coated with Zr metal. The Zr metal coating can be applied by PVD, CVD, ALD and PE-CVD in an inert environment. The coating thickness of Zr metal is 0.5-15 microns. Thereafter, the Zr metal coating is exposed to nitrogen gas and/or a nitrogen containing gas compound and then to carbon and/or a carbon containing gas compound (e.g., methane and/or acetylene gas). The color of the ZrNC will vary depending on the amount of C and N in the coating. Particles of Zr metal can optionally be mixed with nitrogen gas and/or a nitrogen containing gas compound and the carbon and/or a carbon containing gas compound to facilitate in the formation of the ZrNC coating. When Zr metal particles are used, the initial Zr coating layer on the medical device can optionally be eliminated. In one non-limiting embodiment, the enhancement coating composition generally includes 40-95 wt. % Zr (and all values and ranges therebetween), 5-40 wt. % N (and all values and ranges therebetween), and 5-40 wt. % C (and all values and ranges therebetween), 0-2 wt. % O (and all values and ranges therebetween), 0-10 wt. % Re (and all values and ranges therebetween), and 0-20 wt. % Si (and all values and ranges therebetween). In another non-limiting embodiment, the enhancement coating composition generally includes 40-80 wt. % Zr, 5-25 wt. % N, and 5-25 wt. % C, 0-1 wt. % O, 0-8 wt. % Re, and 0-1 wt. % Si.

In accordance with another and/or alternative non-limiting aspect of the present disclosure, the medical device can optionally be configured to be at least partially or fully positioned in the intramedullary canal of a bone. The medical device can optionally be configured to be radially collapsible to a collapsed or crimped state for introduction into the intramedullary canal of a bone and radially expandable to an expanded state for implanting the medical device at a desired location in the intramedullary canal of the bone. The medical device can be optionally formed of a plastically-expandable material that permits crimping of the medical device to a smaller profile for delivery and expansion of the medical device once positioned in the desired location in the intramedullary canal of a bone. The expansion of the crimped frame of the medical device can be by an expansion device such as, but not limited to, a balloon of on a balloon catheter. During the insertion of the medical device into the intramedullary canal of a bone, the medical device can optionally be at least partially surrounded by a flexible sheath. During the insertion of the medical device into the intramedullary canal of a bone, the medical device can optionally be at least partially guided into the intramedullary canal by a guidewire. Prior to, during and/or after the medical device is at least partially positioned in the intramedullary canal of a bone, a portion or all of the intramedullary canal of a bone that includes the medical device can optionally be filled with a column of surgical fluid (e.g., polymer cement, resin, etc.). The medical device is configured to span a fracture site in a bone. The medical device can optionally be introduced into the intramedullary canal of a bone through an incision in the skin and an opening or breach in the fractured bone, along a path which can be along a longitudinal axis of the canal in the bone. Once the medical device is properly positioned in the bone, a cement, adhesive or other surgical fluid can optionally be used to provide fixation and stabilization of the medical device at the bone fracture site. The medical device can be used for fixation and stabilization of long bone fractures; however, the medical device can be used for fixation and stabilization other bones and structures as well. In another non-limiting embodiment, the medical device can be configured to press against or otherwise engage the interior endosteal surface of the bone. The medical device, during its placement and expansion, can be used to assist in moving and/or aligning one or more bone fragments. Re-integration of bone fragments by the medical device can be used to facilitate in the fracture healing process. The medical device can optionally be used to a) aid in the reduction of the fracture by exerting outward forces and pressure from within the intramedullary canal of the fractured bone, and/or b) facilitate the movement or alignment of one or more bone fragments of the fractured bone toward or to a more beneficial or desired location.

In accordance with another and/or alternative non-limiting aspect of the present disclosure, the medical device can be sized and shaped to a) expand with sufficient force to facilitate the movement and re-positioning of one or more bone fragments of a fractured bone that have crushed into or otherwise encroached upon the intramedullary canal (e.g., used to drive such bone fragments radially outward and generally toward a position more aligned with the opposing fracture ends, thereby promoting re-integration of the fragments during the fracture healing process), b) expand to fill gaps or voids in the cortical bone wall or endosteum at or near the fracture site (e.g., if one or more bone fragments have separated away from the fracture site and are not compressed, aligned, or otherwise brought back nearer the fracture site, the medical device can be configured to expand to fill the space once occupied by an absent fragment, and/or c) provide support to the fracture site without requiring additional structures (e.g., sheath, surgical fluid, etc.).

In accordance with another and/or alternative non-limiting aspect of the present disclosure, the medical device can optionally include one or more gripping members disposed on or along the outer surface of the medical device to provide a secure attachment to the inside walls of the intramedullary canal of the bone.

In accordance with another and/or alternative non-limiting aspect of the present disclosure, the medical device has one or more of the following advantages: a) less invasive (e.g., upper extremity 1-3 mm puncture; lower extremity 2-6 mm puncture), b) no screws or plates impinging tendons, muscles etc., c) full internal fixation within the bone, d) preserves bone marrow which is essential to healing, e) insertion sheath for the medical device allows ready outflow for bone marrow getting pushed out and could be reinjected into the bone after insertion of the medical device in the bone, f) reduces risk of pulmonary embolism from bone marrow embolization, g) the medical device can be designed to foreshorten so as to create a built in mechanism for fracture reduction/compression during the expansion of the medical device, h) ends of the medical device can be first expanded to cause fixation of the ends of the medical device in the bone prior to full expansion of the middle portion of the medical device, which expansion of the middle portion of the medical device creates longitudinal compression of the fractured bone due to the foreshortening of the medical device during expansion of the middle portion of the medical device, i) no need for screws to anchor the ends of the medical device in the bone during the expansion of the medical device in the bone (e.g., typical balloon pressures are 10-20 atmospheres ~150 to 300 pounds per square inch when expanding medical device, thus such expansion forces should secure the ends of the medical device within the bone without needs of screws; however, screws can still be used if needed, and/or j) the medical device can include one or more markers to facilitate in the placement of the medical device in the fractured bone.

In accordance with another and/or alternative non-limiting aspect of the present disclosure, a second medical device can be optionally inserted in the interior of a first expanded medical device to increase the strength and/or rigidity of the medical device system at the bone fracture. For example, a first medical device can be inserted and expanded in the bone fracture. Thereafter, a second medical device can optionally be partially or fully inserted into the interior of a portion or all of the expanded first medical device. Generally, the longitudinal length of the expanded second medical device is less (e.g., 20-80% less and all values and ranges therebetween) than the longitudinal length of the expanded first medical device; however, this is not required. After the second medical device is partially or fully inserted into the interior of a portion or all of the expanded first medical device, and thereafter expended, the expanded second medical device generally expands the length of the bone fracture. The second medical device can be configured to a) foreshorten when expanded such that the longitudinal length of the expanded second medical device is at least 15% less (e.g., 15-60% less and all values and ranges therebetween) than a longitudinal length of the crimped or non-expanded second medical device, or b) not or substantially not foreshorten when expanded such that the longitudinal length of the expanded second medical device is no more than 10% less (e.g., 0-10% less and all values and ranges therebetween) than a longitudinal length of the crimped or non-expanded second medical device. When the expanded second medical device is configured to foreshorten, the reduced longitudinal length of the expanded second medical device can facilitate in further drawing the fractured bones together. The use of the expanded second medical device can result in increased rigidity and/or strength in the region of the two expanded medical devices so as to provide additional supported to the fracture region of the bone. The material used to form the first and second medical devices can be the same or different. One or both of the first and second medical devices can include an enhancement coating and/or biological agent.

In another and/or alternative non-limiting aspect of the disclosure, there is method for using a biodegradable or bioabsorbable medical device to promote the fusion of bones, and/or to fix, repair, stabilize and/or fuse of bones and bone fractures. The method includes providing a medical device, which may or may not be an expandable medical device, and wherein at least 10 wt. % (e.g., 10-100% and all values and ranges therebetween) of the medical device is formed of a biodegradable or bioabsorbable metal material. In one non-limiting configuration, the biodegradable or bioabsorbable metal material is formed of a) a molybdenum material wherein the content of molybdenum in the molybdenum material is 80-100% (and all values and ranges therebetween), b) a magnesium material that includes 50 wt. % to 98 wt. % (and all values and ranges therebetween) magnesium, or c) an aluminum material that includes 80 wt. % to 95 wt. % (and all values and ranges therebetween) aluminum. The biodegradable or bioabsorbable metal material is formulated to biodegrade or bioabsorb in a patient after the medical device that includes the biodegradable or bioabsorbable metal material has been implanted in the patient. In another non-limiting configuration, the degradable and/or bioabsorbable metal used to partially or fully form the medical device is formulated to a) exhibit a weight loss of 0.01% to at least 5% (and all values and ranges therebetween) of the degradable and/or bioabsorbable metal after being implanted one year in a patient (e.g., human, mammal, etc.), b) exhibit is a weight loss of 0.05% to at least 8% (and all values and ranges therebetween) of the degradable and/or bioabsorbable metal after being implanted two years in a patient (e.g., human, mammal, etc.), and/or c) exhibit a weight loss of 0.1% to at least 10% (and all values and ranges therebetween) of the degradable and/or bioabsorbable metal after being implanted three years in a patient (e.g., human, mammal, etc.). The method further includes inserting the medical device at a treatment site to promote the fusion of bones, and/or to fix, repair, stabilize and/or fuse of bones and bone fractures. If the medical device is to be used to facilitate in the fusion of adjacently positioned bones, the medical device is inserted at least partially between the two bones to be fused together. The medical device may or may not be expandable. If the medical device is expandable, the medical device can optionally be expanded to facilitate, case, and/or improve the engagement of the medical device with the two bones. An adhesive can be optionally used to facilitate in securing one or more portions of the medical device to one or more portions of one or more bones. If a second expandable medical device is used, the second medical device can be partially or fully inserted inside the first expandable medical device and then expanded. After the one or more medical devices have been inserted or implanted at a treatment site in a patient, the bone about the one or more medical devices begins to grow on and about the one or more medical devices to begin the fusion, fix, repair, and/or stabilization of the bones and/or bone fractures. Also, while the one or more medical devices are in the treatment site, the biodegradable or bioabsorbable metal material used to partially or fully form the medical device begins to slowly biodegrade or be bioabsorbed such that eventually there is at least 10% weight reduction of the biodegradable or bioabsorbable metal material after one or more years after the one or more medical devices have been implanted in the patient.

One non-limiting object of the present disclosure is the provision of an medical device that can be used to promote the fusion of bones and/or be partially or fully inserted into a fractured bone to facilitate in the fixation, repair and stabilization of the fractured bone.

In another and/or alternative non-limiting object of the present disclosure is the provision of an expandable medical device that is partially or fully formed of a degradable and/or bioabsorbable metal that can be used to promote the fusion of bones and/or be partially or fully inserted into a fractured bone to facilitate in the fixation, repair and stabilization of the fractured bone.

In another and/or alternative non-limiting object of the present disclosure is the provision of using a second expandable medical device that is inserted into the interior of a first expanded expandable medical device to increase the strength and/or rigidity of the expandable medical device system at the bone fracture, and wherein both the first and second expandable medical devices are partially or fully formed of a degradable and/or bioabsorbable metal.

In another and/or alternative non-limiting object of the present disclosure is the provision of an expandable medical device for treating a fracture site in a fractured bone having an intramedullary canal; the expandable medical device includes an expandable frame with an open cell configuration; the expandable frame includes a plurality of interconnected struts; the expandable frame has an unexpanded shape and size that enables the expandable frame to be insert into the intramedullary canal; the expandable frame is formed of a degradable and/or bioabsorbable metal; the expandable frame has an expanded shape and size that enables the expandable frame to be secured in the intramedullary canal while traversing a fracture site of the fractured bone; the expandable frame has a longitudinal length that is sufficient to fully span the fracture site; the expandable frame is expandable from a first cross-sectional size in the unexpanded state to a second cross-sectional size in the expanded state; a cross-sectional area of the expandable frame in the second cross-sectional size is larger than a cross-sectional area of the expandable frame in the first cross-sectional size; the longitudinal length of the expandable frame in the unexpanded state is greater than the longitudinal length of the expandable frame in the expanded state; the expandable frame has a side wall that includes a plurality of openings.

In another and/or alternative non-limiting object of the present disclosure is the provision of a medical device that is at least partially coated with a biocompatible material; the biocompatible material includes a) biological agent, b) titanium nitride oxide (TiNOx) or titanium oxynitride (TiNxOy) coating, c) titanium nitride (TiN) coating, d) chromium nitride (CrN) coating, e) diamond-like carbon (DLC) coating, f) zirconium nitride (ZrN) coating, g) zirconium oxide (ZrO2) coating, h) zirconium-nitrogen-carbon (ZrNC) coating, i) zirconium OxyCarbide (ZrOC) coating, and/or j) zirconium oxynitride (ZrNxOy) coating.

In another and/or alternative non-limiting object of the present disclosure is the provision of a method for repairing a bone that is fractured comprising: a) providing a fractured bone that includes first and second bone portions and a fracture site that is located between the first and second bone portions; the fracture site has a fracture site width; each of the first and second bone portions of the fractured bone includes an intramedullary canal; b) providing an expandable medical device for treating the fracture site in the fractured bone; the expandable medical device includes an expandable frame with an open cell configuration; the expandable frame includes a plurality of interconnected struts; the expandable frame has an unexpanded shape and size that enables the expandable frame to be insert into the intramedullary canal; the expandable frame has an expanded shape and size that enables the expandable frame to be secured in the intramedullary canal while traversing a fracture site of the fractured bone; ; the expandable frame is formed of a degradable and/or bioabsorbable metal; the expandable frame has a longitudinal length that is sufficient to fully span the fracture site; the expandable frame is expandable from a first cross-sectional size in the unexpanded state to a second cross-sectional size in the expanded state; a cross-sectional area of the expandable frame in the second cross-sectional size is larger than a cross-sectional area of the expandable frame in the first cross-sectional size; the longitudinal length of the expandable frame in the unexpanded state is greater than the longitudinal length of the expandable frame in the expanded state; the expandable frame has a side wall that includes a plurality of openings; c) inserting the expandable medical device in the intramedullary canal while the expandable medical device in the unexpanded state such that at least a portion of the expandable medical device is positioned in the first and second bone portions and traverses the fracture site; and d) expanding the expandable medical device in the intramedullary canal to the expanded state to repair the fractured bone; and wherein expansion of the expandable medical device causes the first and second bone portions to thereby cause a reduction in the fracture site width, and wherein the expandable frame is that formed of a degradable and/or bioabsorbable metal reduces in weight overtime due to the degradable and/or bioabsorbable properties of the metal material used to form the expandable frame.

In another and/or alternative non-limiting object of the present disclosure is the provision of a method for repairing a bone that is fractured wherein the expandable medical device includes a proximal portion, a distal portion and a mid-portion; and wherein the step of expanding includes expanding the proximal portion and/or a distal portion of the expandable medical device prior to expanding the mid-portion; and wherein prior expansion of the proximal portion and/or a distal portion causes the proximal portion and/or a distal portion to be at least partially anchored in the intramedullary canal prior to expansion of the mid-portion.

In another and/or alternative non-limiting object of the present disclosure is the provision of a method for repairing a bone that is fractured wherein the expandable medical device includes a proximal portion, a distal portion and a mid-portion; and further including the step of securing the proximal portion and/or a distal portion in the intramedullary canal by a) inserting one or more screws or posts into fractured bone to limit movement of the proximal portion and/or a distal portion in the intramedullary canal, and/or b) inserting adhesive and/or cement in the intramedullary canal to limit movement of the proximal portion and/or a distal portion in the intramedullary canal, and wherein the frame, screws and/or post are formed of degradable and/or bioabsorbable metal.

In another and/or alternative non-limiting object of the present disclosure is the provision of a method for repairing a bone that is fractured further including the steps of a) removing at least a portion of bone marrow from the intramedullary canal prior to insertion of the expandable medical device in the intramedullary canal, and b) at least partially inserting at least a portion of the removed bone marrow into the intramedullary canal after the step of expanding the expandable medical device in the intramedullary canal.

In another and/or alternative non-limiting object of the present disclosure is the provision of a method for repairing a bone that is fractured further including the step of using a sheath to facilitate insertion of the expandable medical device into the intramedullary canal; the sheath includes a tubular structure that has a longitudinal cavity; the longitudinal cavity has a size and shape that is configured to enable the expandable medical device in the unexpanded state to move through the longitudinal cavity; at least a portion of the sheath is optionally formed of an elastic material.

In another and/or alternative non-limiting object of the present disclosure is the provision of a method for repairing a bone that is fractured further including the step of using a guidewire to facilitate insertion of a portion of the expandable medical device into the intramedullary canal; the guidewire has sufficient flexibility and stiffness to enable the expandable medical device in the unexpanded state to move through the intramedullary canal and along the guidewire.

In another and/or alternative non-limiting object of the present disclosure is the provision of a method for repairing a bone that is fractured further including the steps of: a) providing a second expandable medical device; b) inserting a second expandable medical device in an interior of the expanded expandable medical device; and c) expanding the second expandable medical device in an interior of the expanded expandable medical device to increase a strength and/or rigidity about a region of the bone fracture, and wherein the second expandable medical device is partially or fully formed of a degradable and/or bioabsorbable metal material.

In another and/or alternative non-limiting object of the present disclosure is the provision of a method for repairing a bone that is fractured wherein a longitudinal length of the second expandable medical device in an expanded state is less than a longitudinal length of the expanded expandable medical device.

In another and/or alternative non-limiting object of the present disclosure is the provision of a method for repairing a bone that is fractured wherein the second expandable medical device is configured to a) foreshorten when expanded such that the longitudinal length of the expanded second expandable medical device is at least 10% less than a longitudinal length of the second expandable medical device in an unexpanded state.

In another and/or alternative non-limiting object of the present disclosure is the provision of a method for repairing a bone that is fractured wherein ends of the second expandable medical device when the second expandable medical device is expanded inside the expandable medical device do not extend beyond ends of the expandable medical device in the expanded state.

In another and/or alternative non-limiting object of the present disclosure is the provision of an expandable device for treating a fracture site in a fractured bone having an intramedullary canal; the expandable device includes an expandable frame; the expandable frame has an unexpanded shape and size that enables the expandable frame to be inserted into the intramedullary canal; the expandable frame has an expanded shape and size that enables the expandable frame to be secured in the intramedullary canal while traversing a fracture site of the fractured bone; the expandable frame has a longitudinal length that is sufficient to fully span the fracture site; the expandable frame is expandable from a first cross-sectional size in the unexpanded state to a second cross-sectional size in the expanded state; a cross-sectional area of the expandable frame in the second cross-sectional size is larger than a cross-sectional area of the expandable frame in the first cross-sectional size; the longitudinal length of the expandable frame in the unexpanded state is greater than the longitudinal length of the expandable frame in the expanded state; the expandable frame has a side wall that includes a one or more openings; the expandable frame is at least partially formed of degradable and/or bioabsorbable metal material; and wherein the expandable frame is optionally at least partially coated with a biocompatible material; the biocompatible material optionally includes a) biological agent, b) titanium nitride oxide (TiNOx) or titanium oxynitride (TiNxOy) coating, c) titanium nitride (TiN) coating, d) chromium nitride (CrN) coating, e) diamond-like carbon (DLC) coating, f) zirconium nitride (ZrN) coating, g) zirconium oxide (ZrO2) coating, h) zirconium-nitrogen-carbon (ZrNC) coating, i) zirconium OxyCarbide (ZrOC) coating, and/or j) zirconium oxynitride (ZrNxOy) coating, and wherein the expandable frame is optionally at least partially coated with a biocompatible material, and wherein the expandable frame optionally has a generally hollow tubular shape, and wherein said biocompatible material optionally includes no more than 0.1 wt. % nickel and/or no more than 0.1 wt. % cobalt, and wherein said metal alloy of said expandable frame optionally includes no more than 0.1 wt. % nickel and/or no more than 0.1 wt. % cobalt, and wherein said biocompatible material is optionally at least partially coated on a metallic adhesion coating and wherein said metallic adhesion coating optionally includes no more than 0.1 wt. % nickel and/or no more than 0.1 wt. % cobalt.

Other objects, advantages, and novel features of the present disclosure will become apparent from the following detailed description of the disclosure when considered in conjunction with the accompanying drawings.

### BRIEF DESCRIPTION OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. Reference may now be made to the drawings, which illustrate various embodiments that the disclosure may take in physical form and in certain parts and arrangement of parts wherein:

FIG. 1 illustrates a non-limiting expandable medical device in accordance with the present disclosure and a list of non-limiting features of the expandable medical device.

FIGS. 2A-2D illustrate a non-limiting method for using the non-limiting expandable medical device in accordance with the present disclosure to repair a bone fracture.

FIG. 3A-3B illustrate the non-limiting expandable medical device in accordance with the present disclosure in an unexpanded and expanded state and also illustrated that the non-limiting expandable medical device in the expanded state has a shorter longitudinal length than the non-limiting expandable medical device in the unexpanded state.

FIG. 4A-4D illustrate a non-limiting method for using the two non-limiting expandable medical devices in accordance with the present disclosure to repair a bone fracture.

### DETAILED DESCRIPTION OF NON-LIMITING EMBODIMENTS

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values) and any ranges therebetween.

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

For the sake of simplicity, the attached figures may not show the various ways (readily discernable, based on this disclosure, by one of ordinary skill in the art) in which the disclosed system, method and apparatus can be used in combination with other systems, methods and apparatuses. Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

The present disclosure is directed to an medical device that can be partially or fully inserted into a fractured bone to facilitate in the fixation, repair, stabilization and/or fusion of bones and bone fractures. The medical device is partially or fully formed of a biodegradable or bioabsorbable metal. The biodegradable or bioabsorbable metal can primarily contain molybdenum, magnesium or aluminum. The medical device can be used to fix, repair and/or stabilize a fractured bone. The medical device can be used in spinal fusion applications for the fusion of one or more bones in a spine. The biodegradable or bioabsorbable metal can primarily contain molybdenum, magnesium or aluminum. The outer surface of the expandable medical device can optionally be coated with an enhancement coating.

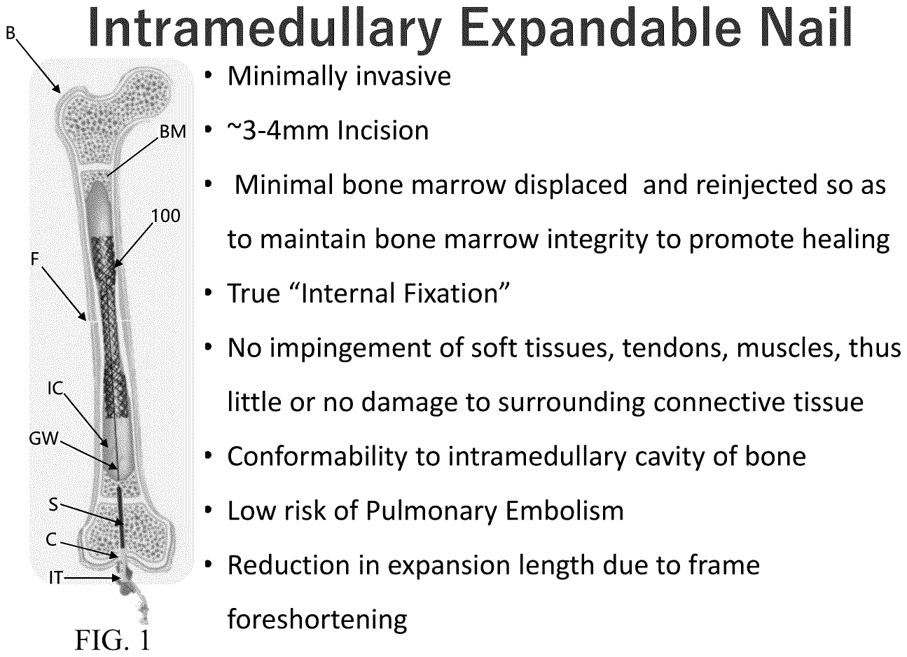

Referring now to FIG. 1, there is illustrated a fractured bone B that includes the expandable medical device 100 positioned in the intramedullary canal IC of the fractured bone B. The medical device 100 is positioned in the intramedullary canal IC such that the medical device 100 overlies the fracture zone F of the fractured bone B. The bone marrow BM has been partially or fully removed in the intramedullary canal IC prior to the insertion of the medical device 100 into the intramedullary canal IC. A guidewire GW is illustrated that can optionally be used to facilitate in guiding the medical device 100 during insertion into the intramedullary canal IC. The base of the fractured bone includes a channel C that has been cut into the fracture bone so that the medical device 100 can be inserted into the intramedullary canal IC. An insertion tool IC is used to inert the medical device 100 into the intramedullary canal IC. The insertion tool IC can optionally include a sheath S that is positioned in channel C and can be used to facilitate in the insertion of the medical device 100 though channel C and into the intramedullary canal IC.

FIG. 1 lists several non-limiting advantages of the expandable medical device 100, namely:

The use of the expandable medical device 100 is minimally invasive. Generally, only a 3-4 mm incision is required in the bone to insert the medical device 100 into the intramedullary canal IC of the fractured bone B. Prior art intramedullary nail generally required an incision into the bone of about 15 mm. The significantly smaller incision in the fractured bone results in improved healing rates of the fractured bone, reduced incidence of damage to the bone during the creation of the incision,

The minimizes the loss of bone marrow during the fracture repair procedure since removed or displaced bone marrow is reinjected into the intramedullary canal after the medical device has been inserted into the intramedullary canal so as to maintain bone marrow integrity and to promote healing of the fractured bone.

The use of the medical device 100 provides for true “Internal Fixation” of the fracture without the need for external devices (e.g., screws, exterior support structures, etc.). The “Internal Fixation” feature of the medical device also results in no impingement of soft tissues, tendons, muscles about the fractured bone, thus little or no damage to surrounding connective tissue about the fracture bone during the repair of the fractured bone.

The use of the medical device 100 provides improved conformity to the shape of the intramedullary canal of the fractured bone as compared to a rigid rod. It is common that the shape of the intramedullary canal of the fractured bone is not linear, especially when the bone is fractured. The flexibility of the frame of the medical device allow the frame to bend during the insertion of the medical device into the intramedullary canal. As such, the flexible frame of the medical device is able to better conform to a non-linear shaped intramedullary canal of the fractured bone, thus resulting in a) improved fracture repair, and b) reduced damage to the intramedullary canal of the fractured bone during insertion of the medical device into the intramedullary canal.

The use of the medical device 100 to repair a fractured bone can result in a lower risk of Pulmonary Embolism.

The medical device 100 is configured to reduce in longitudinal length when the medical device is expanded due to frame foreshortening. Such reduction in longitudinal length of the medical device results in drawing together of the fractured potions of the fractured bone, thereby improving fracture repair.

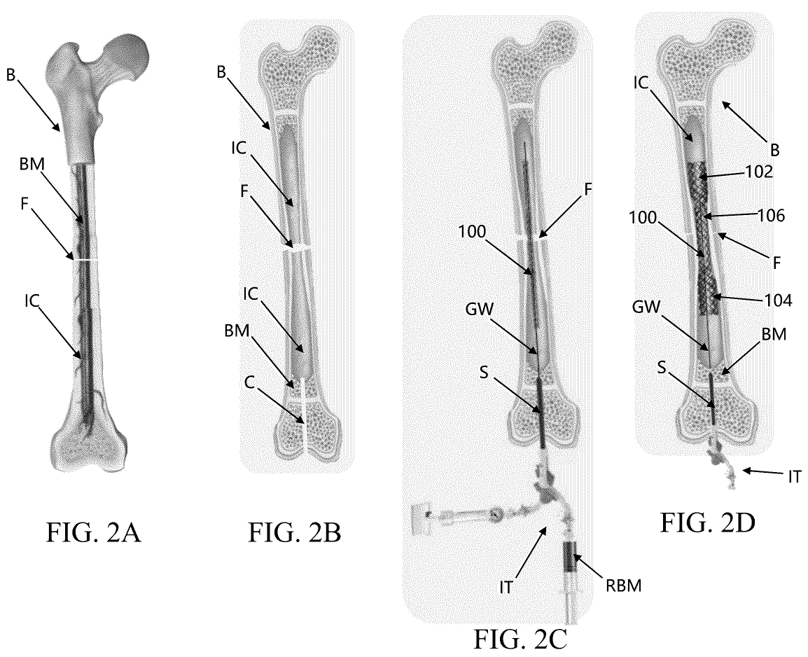

Referring now to FIGS. 2A-2D, there is illustrated a non-limiting method for repairing a fractured bone B by use of the medical device 100. FIG. 2A illustrates a fractured bone B that includes a fracture zone F. The fractured bone F includes an intramedullary canal IC that includes bone marrow BM and other tissue and/or blood vessels. Referring now to FIG. 2A, a channel C in one end of the fractured bone B has been formed. The diameter of the channel can be as small as 2 mm and is typically 3-6 mm (and all values and ranges therebetween). After the channel C is formed, a portion of all of the bone marrow BM and other tissue and/or blood vessels in the intramedullary canal IC can be temporarily removed as illustrated in FIG. 2B. The process to remove the bone marrow BM and other tissue and/or blood vessels in the intramedullary canal IC is known in the art and will not be described herein.

Referring now to FIG. 2C, once the bone marrow BM and other tissue and/or blood vessels in the intramedullary canal IC has been sufficiently removed, an insertion tool IT is used to facilitate in the insertion of the medical device 100 into the intramedullary canal IC. The insertion tool IT can optionally include the use of a sheath S and/or a guidewire GW to facilitate in the insertion of the medical device 100 into the intramedullary canal IC. The sheath is illustrated as being inserted into channel C. The sheath S can optionally be formed of an expandable material that can expand as the medical device 100 is moved through the internal channel of the sheath and into the intramedullary canal IC. The sheath S can be formed of a flexible polymer material. The sheath S can optionally include a shape memory material that cause the sheath to return to its original shape or near original shape as or after the medical device 100 has passed through the internal channel of the sheath. The sheath S is configured to inhibit or prevent damage to the bone marrow that is located about channel C as the medical device 100, while in the crimped or unexpanded state, is passed through the channel C. The sheath S is also configured to inhibit or prevent damage to the medical device 100 as it is passed through the channel C (e.g., front end of expandable medical device gets caught on the bone marrow about the channel, thus bending or otherwise damaging the medical device, etc.). Generally, no more than 50% (e.g., 0-50% and all values and ranges therebetween) of the longitudinal length of the sheath S is inserted into the portion of the intramedullary canal IC where a portion of all of the bone marrow BM and other tissue and/or blood vessels in the intramedullary canal IC has been removed. As illustrated in FIGS. 2C and 2D, the proximal end of sheath S is spaced from the portion of the intramedullary canal IC where a portion of all of the bone marrow BM and other tissue and/or blood vessels in the intramedullary canal IC has been removed; however, this is not required.

The insertion tool IT can optionally include the use of a guidewire GW to facilitate in the insertion of the medical device 100 into the intramedullary canal IC. The guidewire GW is illustrated as being inserted through the channel C and into the portion of the intramedullary canal IC where a portion of all of the bone marrow BM and other tissue and/or blood vessels in the intramedullary canal IC has been removed. If a sheath S is used, the guidewire GW is insert through the internal channel of the sheath S. As illustrated in FIG. 2C, the guidewire GW extends into at least 50% (e.g., 50-100% and all values and ranges therebetween) of the longitudinal length of the portion of the intramedullary canal IC where a portion of all of the bone marrow BM and other tissue and/or blood vessels in the intramedullary canal IC has been removed.

After the optional sheath S and guidewire GW are positioned in the fractured bone B, the medical device 100 is inserted, while in its crimped or unexpanded state, into the portion of the intramedullary canal IC where a portion of all of the bone marrow BM and other tissue and/or blood vessels in the intramedullary canal IC has been removed. When a sheath S is used, the medical device 100 is inserted fully through the internal channel of the sheath S. When a guidewire GW is used, the medical device 100 is inserted about the guidewire GW and then guided along the guidewire and into the intramedullary canal IC until the medical device 100 is properly positioned in the intramedullary canal IC. Generally, the intramedullary canal IC is not inserted past the end of the guidewire GW prior to the expansion of the medical device 100 in the intramedullary canal IC.

Once the medical device 100 is positioned in the intramedullary canal IC, at least a portion of the frame or body of the medical device 100 is expanded form a crimped state to an expanded state in the intramedullary canal IC. As illustrated in FIG. 2D, the posterior portion 102 and anterior portion 104 of the medical device 100 are expanded in the intramedullary canal IC. Such expansion of the posterior and anterior portions 102, 104 of the medical device 100 in the intramedullary canal IC result in the anchoring of the posterior portion 102 of the medical device 100 in one section of the fractured bone B and anchoring of the anterior portion 104 of the medical device 100 in the other section of the fractured bone B. Once both the posterior and anterior portions 102, 104 of the expandable medical device 100 are properly anchored in the intramedullary canal IC, the medical device 100 can optionally be further expanded in the expandable medical device 100 in the intramedullary canal IC. The further expansion of the medical device 100 can occur at the posterior and anterior portions 102, 104 of the medical device 100, and/or in the mid-portion 106 of the medical device 100. The expansion of the posterior and anterior portions 102, 104 of the medical device 100 can be simultaneous or sequential (e.g., partial or full expansion of posterior portion prior to partial or full expansion of anterior portion, partial or full expansion of anterior portion prior to partial or full expansion of posterior portion, etc.). The mid-portion 106 can be optionally expanded after the full expansion of the posterior and anterior portions 102, 104 of the medical device 100.

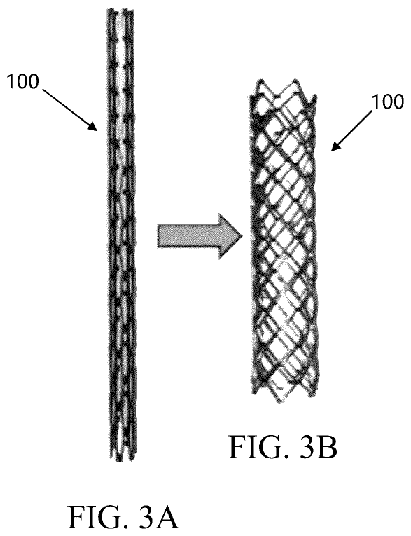

The frame or body of the medical device 100 can be configured to result in foreshortening of the longitudinal length of the medical device when the medical device is expanded from the crimped to the expanded state. Referring now to FIGS. 3A and 3B, there is illustrated a non-limiting configuration of the frame or body of the medical device 100 that is configured to foreshorten when expanded. The frame or body has an open cell configuration and is formed of a plurality of interconnecting struts and/or posts. The interconnecting struts and/or posts can be configured to form a variety of patterns (e.g., zig-zag pattern, saw-tooth pattern, triangular pattern, polygonal pattern, oval pattern, etc.). One or more of the interconnecting struts and/or posts can have the same or different thicknesses and/or cross-sectional shape and/or cross-sectional area. FIG. 3A illustrates the medical device 100 in the crimped or expanded state. FIG. 3B illustrates the medical device 100 in the expanded state. The longitudinal length of the medical device 100 in the expanded state is illustrates as being less than the longitudinal length medical device 100 in the crimped or unexpanded state. In one non-limiting embodiment, the longitudinal length of the medical device 100 in the expanded state is at least 5% (e.g., 5-60% and all values and ranges therebetween) less than the longitudinal length medical device 100 in the crimped or unexpanded state. An inflation device (e.g., expandable balloon, etc.) can be used to expand the medical device 100.

Referring now to FIGS. 2C and 2D, when the medical device 100 is expanded and the longitudinal length of the medical device 100 is reduced, the reduction in longitudinal length of the medical device 100 causes the fractured portions of the fractured bone B to be drawn together thereby facilitating in the repair of the fractured bone B.

The frame or body of the medical device 100 is generally partially or fully formed of a a) molybdenum material that includes 80 wt. % to 100 wt. % (and all values and ranges therebetween) molybdenum, b) a magnesium material that includes 50 wt. % to 98 wt. % (and all values and ranges therebetween) magnesium, or c) an aluminum material that includes 80 wt. % to 95 wt. % (and all values and ranges therebetween) aluminum. In one non-limiting embodiment, the degradable and/or bioabsorbable metal is formed of 95-100 wt. % (and all values and ranges therebetween) metals, and 0-5 wt. % (and all values and ranges therebetween) of a combined weight of carbon, oxygen, phosphorous, sulfur, hydrogen, nitrogen, carbides, nitrides, and compounds that include oxygen, nitrogen, hydrogen, phosphorous, sulfur, and/or carbon. In another non-limiting embodiment, the degradable and/or bioabsorbable metal used to partially or fully form the medical device includes 80 wt. % to 100 wt. % (and all values and ranges therebetween) of a magnesium material that includes 50 wt. % to 98 wt. % (and all values and ranges therebetween) magnesium, and 0.05 wt. % to 49.5 wt. % (and all values and ranges therebetween) of one or more metals selected from the group consisting of aluminum, bismuth, boron, calcium, cobalt, copper, gallium, manganese, nickel, tin, zinc, and zirconium. In another non-limiting formulation, 90-100 wt. % (and all values and ranges therebetween) of the aluminum material includes metals selected from aluminum, bismuth, calcium, gallium, magnesium, and tin.

The frame or body of the medical device 100 can be optionally fully or partially coated with an enhancement coating. Non-limiting enhancement coatings that can be applied to a portion or all of the expandable medical device includes chromium nitride (CrN), diamond-like carbon (DLC), titanium nitride (TiN), titanium nitride oxide (TiNOx) or titanium oxynitride (TiNxOy), zirconium nitride (ZrN), zirconium oxide (ZrO2), zirconium-nitrogen-carbon (ZrNC), zirconium OxyCarbide (ZrOC), zirconium oxynitride (ZrNxOy) [e.g., cubic ZrN: O, cubic ZrO2:N, tetragonal ZrO2:N, and monoclinic ZrO2: N phase coatings], and combinations of such coatings. In another non-limiting embodiment, the thickness of the enhancement coating is greater than 1 nanometer (e.g., 2 nanometers to 100 microns and all values and ranges therebetween). In one non-limiting configuration, the 50-100% (and all values and ranges therebetween) of the outer surface of the frame or body of the medical device 100 is coated with titanium nitride oxide (TiNOx) or titanium oxynitride (TiNxOy) and/or zirconium oxynitride (ZrNxOy), and wherein the enhancement coating is formulated to promote nitric oxide formation on and/or about the implanted medical device.

Referring again to FIGS. 2C and 2D, after the medical device 100 is expanded in the intramedullary canal IC, a portion of all of the removed bone marrow (RBM) that was removed from the intramedullary canal IC can optionally be reinserted into the intramedullary canal IC via an insertion device (e.g., syringe, etc.). Also, once the medical device 100 is properly positioned and expanded in the intramedullary canal IC of the fractured bone B, a cement, adhesive or other surgical fluid can optionally be used to provide fixation and stabilization of the medical device 100 in the intramedullary canal IC. The cement, adhesive or other surgical fluid, when used, can be inserted in the intramedullary canal IC prior to, during or after the optional insertion of the removed bone marrow (RBM) into the intramedullary canal IC. Generally, the guidewire (GW), when used is partially or fully removed from the intramedullary canal IC prior to the optional insertion of the cement, adhesive or other surgical fluid and/or the removed bone marrow (RBM) into the intramedullary canal IC. The sheath S is removed from the intramedullary canal IC prior to, during or after the full expansion of the medical device 100 in the intramedullary canal IC, and/or the optional insertion of the cement, adhesive or other surgical fluid and/or the removed bone marrow (RBM) into the intramedullary canal IC. After the insertion tool IT is removed from channel C, channel C can optionally be sealed by a sealing material (e.g., cement, adhesive or other surgical fluid, bone fragments, etc.) after the full expansion of the medical device 100 in the intramedullary canal IC, and the optional insertion of the cement, adhesive or other surgical fluid and/or the removed bone marrow (RBM) into the intramedullary canal IC.

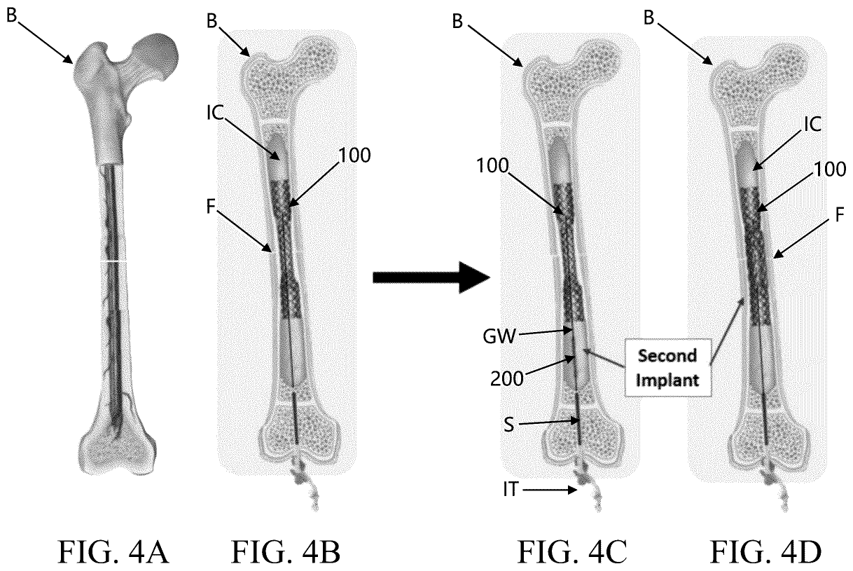

Referring now to FIGS. 4A-4D, there is illustrated a modified procedure to the bone repair procedure illustrated in FIGS. 2A-2D. The modified procedure includes the procedure discussed above with regard to FIGS. 2A-2D, but includes the use of a second medical device 200. A second medical device 200 can be optionally inserted in the interior of a first medical device 100 to increase the strength and/or rigidity of the medical device system at the fracture F of the fractured bone B. For example, the first medical device 100 can be inserted and expanded in the intramedullary canal IC as discussed above with respect to FIGS. 2A-2D. Thereafter, a second medical device 200 can be inserted into the interior of the expanded first medical device 200. Generally, the longitudinal length of the expanded second medical device 200 is less (e.g., 20-80% less and all values and ranges therebetween) than the longitudinal length of the expanded first medical device 100.

Generally, the longitudinal length of the unexpanded second medical device 200 is equal to or less (e.g., 20-80% less and all values and ranges therebetween) than the longitudinal length of the unexpanded first medical device 100. In one non-limiting configuration, the longitudinal length of the unexpanded second medical device 200 is less than the longitudinal length of the unexpanded first medical device 100 such that when the unexpanded second medical device 200 is inserted inside the expanded first medical device 100, the ends of the unexpanded second medical device 200 are spaced inwardly from the ends of the expanded first medical device 100. In another non-limiting configuration, when the first and second medical devices 100, 200 are expanded, the ends of the expanded second medical device 200 are spaced inwardly from the ends of the expanded first medical device 100.

As illustrated in FIG. 4D, the expanded second medical device 200 traverses the length of the bone fracture. Generally, 10-70% of the longitudinal length (and all values and ranges therebetween) of one side of the expanded second medical device 200 is located on one side of the fracture F, and 10-70% of the longitudinal length (and all values and ranges therebetween) of the other side of the expanded second expandable medical device 200 is located on the other side of the fracture F. As illustrated in FIG. 4C, the second medical device 200 is inserted into the interior of the expanded first medical device 100 prior to the expansion of the second medical device 200. The second medical device 200 can be configured to a) foreshorten when expanded such that the longitudinal length of the expanded second medical device is at least 10% less (e.g., 10-60% less and all values and ranges therebetween) than a longitudinal length of the crimped or non-expanded second medical device 200, or b) not or substantially not foreshorten when expanded such that the longitudinal length of the expanded second medical device is no more than 10% less (e.g., 0-10% less and all values and ranges therebetween) than a longitudinal length of the crimped or non-expanded second medical device 200. When the expanded second medical device 200 is configured to foreshorten, the reduced longitudinal length of the expanded second medical device 200 can facilitate in further drawing the fractured portion of the fractured bone B together. The use of the expanded second medical device 200 results in increased rigidity and/or strength in the region of the two expanded expandable medical devices 100, 200 so as to provide additional supported to the fracture region of the fractured bone B. The material used to form the first and second medical devices 100, 200 can be the same or different. In one non-limiting configuration, both the first and second medical devices 100, 200 are partially or fully formed of a degradable and/or bioabsorbable metal material. One or both of the first and second devices 100, 200 can include an enhancement coating and/or biological agent.

After the second medical device 200 has been expanded, a portion of all of the removed bone marrow (RBM) that was removed from the intramedullary canal IC can optionally be reinserted into the intramedullary canal IC via an insertion device (e.g., syringe, etc.). Also, once the medical devices 100, 200 are properly positioned and expanded in the intramedullary canal IC of the fractured bone B, a cement, adhesive or other surgical fluid can optionally be used to provide fixation and stabilization of the medical devices 100, 200 in the intramedullary canal IC. The cement, adhesive or other surgical fluid, when used, can be inserted in the intramedullary canal IC prior to, during or after the optional insertion of the removed bone marrow (RBM) into the intramedullary canal IC. Generally, the guidewire (GW), when used is partially or fully removed from the intramedullary canal IC prior to the optional insertion of the cement, adhesive or other surgical fluid and/or the removed bone marrow (RBM) into the intramedullary canal IC. The sheath S is removed from the intramedullary canal IC prior to, during or after the full expansion of the medical devices 100, 200 in the intramedullary canal IC, and/or the optional insertion of the cement, adhesive or other surgical fluid and/or the removed bone marrow (RBM) into the intramedullary canal IC. After the insertion tool IT is removed from channel C, channel C can optionally be sealed by a sealing material (e.g., cement, adhesive or other surgical fluid, bone fragments, etc.) after the full expansion of the medical devices 100, 200 in the intramedullary canal IC, and the optional insertion of the cement, adhesive or other surgical fluid and/or the removed bone marrow (RBM) into the intramedullary canal IC.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall therebetween.

To aid the Patent Office and any readers of this application and any resulting patent in interpreting the claims appended hereto, applicants do not intend any of the appended claims or claim elements to invoke 35 U.S.C. 112(f) unless the words “means for” or “step for” are explicitly used in the particular claim.

What is claimed:

1. A medical device for treating a fracture site in a fractured bone or for the fusing together of two adjacently positioned bones; said medical device includes a body portion formed of a metal material that is formulated to be degradable or bioabsorable in a patient after the medical device has been implanted in the patient.

2. The medical device as defined in claim 1, wherein said metal material that is formulated to be degradable or bioabsorable includes a) a molybdenum material that includes 80 wt. % to 100 wt. % molybdenum, b) a magnesium material that includes 50 wt. % to 98 wt. % magnesium, c) an aluminum material that includes 80 wt. % to 95 wt. % aluminum, or d) a zinc material that includes 80 wt. % to 99 wt. % zinc.

3. The medical device as defined in claim 2, wherein said metal material that is formulated to be degradable or bioabsorable includes 95-100 wt. % metal, and 0-5 wt. % of a combined weight of carbon, oxygen, phosphorous, sulfur, hydrogen, nitrogen, carbides, nitrides, and compounds that include oxygen, nitrogen, hydrogen, phosphorous, sulfur, and/or carbon.

4. The medical device as defined in claim 2, wherein said metal material that is formulated to be degradable or bioabsorable includes 99.9-100 wt. % metal, and 0-0.1 wt. % of a combined weight of carbon, oxygen, phosphorous, sulfur, hydrogen, nitrogen, carbides, nitrides, and compounds that include oxygen, nitrogen, hydrogen, phosphorous, sulfur, and/or carbon.

5. The medical device as defined in claim 2, wherein said metal material that is formulated to be degradable or bioabsorable includes said molybdenum material.

6. The medical device as defined in claim 2, wherein said metal material that is formulated to be degradable or bioabsorable includes said magnesium material that includes 50-98 wt. % magnesium and 0.05-49.5 wt. % of one or more metals selected from the group consisting of aluminum, bismuth, boron, calcium, cobalt, copper, gallium, manganese, nickel, rare earth metal, tin, yttrium, zinc, and zirconium, and wherein 90-100 wt. % of said magnesium material includes metals selected from magnesium, aluminum, bismuth, boron, calcium, cobalt, copper, gallium, manganese, nickel, rare earth metal, tin, yttrium, zinc, and zirconium.

7. The medical device as defined in claim 2, wherein said metal material that is formulated to be degradable or bioabsorable includes said aluminum material that includes 80-95 wt. % aluminum and 5-20wt. % of one or more metals selected from the group consisting of bismuth, calcium, gallium, magnesium, rare earth metal, and tin, and wherein 90-100 wt. % of said aluminum material includes metals selected from aluminum, bismuth, calcium, gallium, magnesium, rare earth metal, and tin.

8. The medical device as defined in claim 2, wherein said metal material that is formulated to be degradable or bioabsorable includes said zinc material that includes 80-99.9 wt. % zinc and 0.1-20 wt. % of one or more metals selected from the group consisting of bismuth, copper, calcium, lithium, magnesium, rare earth metal, silver, strontium, and tin, and wherein 90-100 wt. % of said zinc material includes metals selected from zinc, bismuth, copper, calcium, lithium, magnesium, rare earth metal, silver, strontium, and tin.

9. The medical device as defined in claim 1, wherein said metal material is formulated to result one or more of a) a weight loss of 0.01% to at least 5% after being implanted one year in a patient, b) a weight loss of 0.05% to at least 8% after being implanted two years in a patient, c) a weight loss of 0.1% to at least 10% after being implanted three years in a patient, or 4) a weight loss of at least 30% after being implanted ten years in a patient.

10. The medical device as defined in claim 1, wherein said body portion is or includes an expandable frame.

11. The medical device as defined in claim 10, wherein said expandable frame has an unexpanded shape and size that enables said expandable frame to be inserted into an intramedullary canal of the bone; said expandable frame has an unexpanded state that enables said expandable frame to be inserted into said intramedullary canal; said expandable frame has an expanded state that enables said expandable frame to be secured in the intramedullary canal; said expandable frame is expandable from a first cross-sectional size in said unexpanded state to a second cross-sectional size in said expanded state; a cross-sectional area of said expandable frame in said second cross-sectional size is larger than a cross-sectional area of said expandable frame in said first cross-sectional size; said expandable frame has a side wall that includes one or more openings in a frame wall of said expandable frame.

12. The expandable device as defined in claim 1, wherein said body portion is at least partially coated with a biocompatible material; said biocompatible material includes a) biological agent, b) titanium nitride oxide (TiNOx) or titanium oxynitride (TiNxOy) coating, c) titanium nitride (TiN) coating, d) chromium nitride (CrN) coating, e) diamond-like carbon (DLC) coating, f) zirconium nitride (ZrN) coating, g) zirconium oxide (ZrO2) coating, h) zirconium-nitrogen-carbon (ZrNC) coating, i) zirconium OxyCarbide (ZrOC) coating, and/or j) zirconium oxynitride (ZrNxOy) coating.

13. A method for repairing a fracture site in a fractured bone or for fusing together two adjacently positioned bones comprising:
providing a) a fractured bone that includes first and second bone portions and a fracture site that is located between said first and second bone portions; said fracture site has a fracture site width; each of said first and second bone portions of said fractured bone includes an intramedullary canal, b) first and second bones that are to be fused together;
providing a medical device; said medical device includes a body portion formed of a metal material that is formulated to be degradable or bioabsorable in a patient after the medical device has been implanted in the patient; and
inserting said medical either a) between said first and second bones to facilitate in said fusion together of said first and second bone, or b) connecting said medical device to said first and second bone portions to facilitate in said repairing of said fractured bone, and
wherein said metal material on said body portion of said medical device degrades or is bioabsorbed such that the weight of said metal material reduces by at least 0.01 wt. % per year after said medical device has been implanted in a patient.

14. The method as defined in claim 13, wherein said body of said medical device includes a first expandable frame; said first expandable frame has an unexpanded shape and size that enables said first expandable frame to be inserted into an intramedullary canal of said first and second bone portions; said first expandable frame has an expanded shape and size that enables said first expandable frame to be secured in said intramedullary canal of said first and second bone portions while traversing a fracture site of said fractured bone; said first expandable frame has a longitudinal length that is sufficient to fully span said fracture site; said first expandable frame is expandable from a first cross-sectional size in said unexpanded state to a second cross-sectional size in said expanded state; a cross-sectional area of said first expandable frame in said second cross-sectional size is larger than a cross-sectional area of said first expandable frame in said first cross-sectional size; said longitudinal length of said first expandable frame in said unexpanded state is greater than said longitudinal length of said first expandable frame in said expanded state; said first expandable frame has a side wall that includes a one or more openings; said method further includes the steps of:
inserting said first expandable frame in said intramedullary canal of said first and second bone portions while said first expandable frame in said unexpanded state such that at least a portion of said first expandable frame is positioned in said first and second bone portions and traverses said fracture site; and
expanding said first expandable frame in said intramedullary canal of said first and second bone portions to said expanded state to repair said fractured bone; and
wherein expansion of said first expandable frame causes said first and second bone portions to move together and thereby cause a reduction in a fracture site width.

15. The method as defined in claim 14, wherein said first expandable frame includes a proximal portion, a distal portion and a mid-portion; and wherein said step of expanding includes expanding said proximal portion and/or a distal portion of said first expandable frame prior to expanding a mid-portion of said first expandable frame; and wherein prior expansion of said proximal portion and/or a distal portion causes said proximal portion and/or a distal portion to be at least partially anchored in said intramedullary canal of said first and second bone portions prior to expansion of said mid-portion.

16. The method as defined in claim 15, further including the step of securing said proximal portion and/or a distal portion of said first expandable frame in said intramedullary canal in said first and/or second bone portions by a) inserting one or more screws or posts into fractured bone to limit movement of said proximal portion and/or a distal portion in said intramedullary canal of said first and/or second bone portions, and/or b) inserting adhesive and/or cement in said intramedullary canal of said first and/or second bone portions to limit movement of said proximal portion and/or a distal portion in said intramedullary canal of said first and/or second bone portions.

17. The method as defined in claim 14, further including the steps of a) removing at least a portion of bone marrow from said intramedullary canal of said first and second bone portions prior to insertion of said first expandable frame in said intramedullary canal of said first and second bone portions, and b) at least partially inserting at least a portion of said removed bone marrow into said intramedullary canal of at least one of said first and second bone portions after said step of expanding said first expandable frame in said intramedullary canal of said first and second bone portions.

18. The method as defined in claim 14, further including the step of using a sheath to facilitate insertion of said first expandable frame into said intramedullary canal of at least one of said first and second bone portions; said sheath includes a tubular structure that has a longitudinal cavity; said longitudinal cavity has a size and shape that is configured to enable said first expandable frame in said unexpanded state to move through said longitudinal cavity; at least a portion of said sheath is optionally formed of an elastic material.

19. The method as defined in claim 14, further including the step of using a guidewire to facilitate insertion of a portion of said expandable frame into said intramedullary canal of at least one of said first and second bone portions; said guidewire has sufficient flexibility and stiffness to enable said first expandable frame in said unexpanded state to move through said intramedullary canal of at least one of said first and second bone portions and along said guidewire.

20. The method as defined in claim 14, further including the steps of:
providing a second expandable device; said second expandable device includes a second expandable frame with at least one opening; said second expandable frame, when oriented in an unexpanded shape and size, enables said second expandable device to be insert into said intramedullary canal of said first and second bone portions; said second expandable frame is configured to be expanded to an expanded shape and size; said second expandable frame has a longitudinal length that is sufficient to fully span said fracture site; said second expandable frame is expandable from a first cross-sectional size in said unexpanded state to a second cross-sectional size; a cross-sectional area of said second expandable frame in said second cross-sectional size is larger than a cross-sectional area of said second expandable frame in said first cross-sectional size; said longitudinal length of said second expandable frame in said unexpanded state is greater than said longitudinal length of said second expandable frame in said expanded state; said second expandable frame has a side wall that includes a plurality of openings; second expandable frame is formed of a metal material that is formulated to be degradable or bioabsorable in a patient after the medical device has been implanted in the patient;
inserting a second expandable frame in an interior of said expanded first expandable frame; and
expanding said second expandable device in an interior of said first expanded expandable frame to increase a strength and/or rigidity about a region of said bone fracture.

21. The method as defined in claim 20, wherein a longitudinal length of said second expandable frame in an expanded state is less than a longitudinal length of said expanded first expandable frame.

22. The method as defined in claim 20, wherein said second expandable frame is configured to a) foreshorten when expanded such that said longitudinal length of said expanded second expandable frame is at least 10% less than a longitudinal length of said second expandable frame in an unexpanded state.

23. The method as defined in claim 20, wherein ends of said second expandable frame when said second expandable frame is expanded inside said first expandable frame do not extend beyond ends of said first expandable frame when said first expandable frame in said expanded state.

24. The method as defined in claim 13, wherein said metal material that is formulated to be degradable or bioabsorable includes a) a molybdenum material that includes 80 wt. % to 100 wt. % molybdenum, b) a magnesium material that includes 50 wt. % to 98 wt. % magnesium, c) an aluminum material that includes 80 wt. % to 95 wt. % aluminum, or d) a zinc material that includes 80 wt. % to 99 wt. % zinc.

25. The method as defined in claim 24, wherein said metal material that is formulated to be degradable or bioabsorable includes 95-100 wt. % metal, and 0-5 wt. % of a combined weight of carbon, oxygen, phosphorous, sulfur, hydrogen, nitrogen, carbides, nitrides, and compounds that include oxygen, nitrogen, hydrogen, phosphorous, sulfur, and/or carbon.

26. The method as defined in claim 24, wherein said metal material that is formulated to be degradable or bioabsorable includes 99.9-100 wt. % metal, and 0-0.1 wt. % of a combined weight of carbon, oxygen, phosphorous, sulfur, hydrogen, nitrogen, carbides, nitrides, and compounds that include oxygen, nitrogen, hydrogen, phosphorous, sulfur, and/or carbon.

27. The method as defined in claim 24, wherein said metal material that is formulated to be degradable or bioabsorable includes said molybdenum material.

28. The method as defined in claim 24, wherein said metal material that is formulated to be degradable or bioabsorable includes said magnesium material that includes 50-98 wt. % magnesium and 0.05-49.5 wt. % of one or more metals selected from the group consisting of aluminum, bismuth, boron, calcium, cobalt, copper, gallium, manganese, nickel, rare earth metal, tin, yttrium, zinc, and zirconium, and wherein 90-100 wt. % of said magnesium material includes metals selected from magnesium, aluminum, bismuth, boron, calcium, cobalt, copper, gallium, manganese, nickel, rare earth metal, tin, yttrium, zinc, and zirconium.

29. The method as defined in claim 24, wherein said metal material that is formulated to be degradable or bioabsorable includes said aluminum material that includes 80-95 wt. % aluminum and 5-20 wt. % of one or more metals selected from the group consisting of bismuth, calcium, gallium, magnesium, rare earth metal, and tin, and wherein 90-100 wt. % of said aluminum material includes metals selected from aluminum, bismuth, calcium, gallium, magnesium, rare earth metal, and tin.

30. The method as defined in claim 24, wherein said metal material that is formulated to be degradable or bioabsorable includes said zinc material that includes 80-99.9 wt. % zinc and 0.1-20 wt. % of one or more metals selected from the group consisting of bismuth, copper, calcium, lithium, magnesium, rare earth metal, silver, strontium, and tin, and wherein 90-100 wt. % of said zinc material includes metals selected from zinc, bismuth, copper, calcium, lithium, magnesium, rare earth metal, silver, strontium, and tin.

31. The method as defined in claim 13, wherein said metal material is formulated to result one or more of a) a weight loss of 0.01% to at least 5% after being implanted one year in a patient, b) a weight loss of 0.05% to at least 8% after being implanted two years in a patient, c) a weight loss of 0.1% to at least 10% after being implanted three years in a patient, or 4) a weight loss of at least 30% after being implanted ten years in a patient.

32. The method as defined in claim 13, wherein said body portion is at least partially coated with a biocompatible material; said biocompatible material includes a) biological agent, b) titanium nitride oxide (TiNOx) or titanium oxynitride (TiNxOy) coating, c) titanium nitride (TiN) coating, d) chromium nitride (CrN) coating, e) diamond-like carbon (DLC) coating, f) zirconium nitride (ZrN) coating, g) zirconium oxide (ZrO2) coating, h) zirconium-nitrogen-carbon (ZrNC) coating, i) zirconium OxyCarbide (ZrOC) coating, and/or j) zirconium oxynitride (ZrNxOy) coating.

33. The method as defined in claim 20, wherein said second expandable frame is at least partially coated with a biocompatible material; said biocompatible material includes a) biological agent, b) titanium nitride oxide (TiNOx) or titanium oxynitride (TiNxOy) coating, c) titanium nitride (TiN) coating, d) chromium nitride (CrN) coating, e) diamond-like carbon (DLC) coating, f) zirconium nitride (ZrN) coating, g) zirconium oxide (ZrO2) coating, h) zirconium-nitrogen-carbon (ZrNC) coating, i) zirconium OxyCarbide (ZrOC) coating, and/or j) zirconium oxynitride (ZrNxOy) coating.

34. The method as defined in claim 20, wherein said metal material used to at least partially form said second expandable frame is the same as said metal material used to at least partially form said first expandable frame.
